(19) United States
(12) Patent Application Publication
(10) Pub. No.: US 2009/0192360 A1
(43) Pub. Date: Jul. 30, 2009

(54) ATRAUMATIC SURGICAL RETRACTION AND HEAD-CLAMPING DEVICE

(76) Inventors: Edward Allen Riess, Cincinnati, OH (US); Jeffery L. Stambough, Cincinnati, OH (US); Carroll E. Weller, Mason, OH (US)

Correspondence Address: Edward Allen Riess, 7962 Quebec Court, Cincinnati, OH, 45241-1359, US

(21) Appl. No.: 12/361,460

(22) Filed: Jan. 28, 2009

Related U.S. Application Data
(60) Provisional application No. 61/062,482, filed on Jan. 28, 2008.

(51) Int. Cl. A61B 1/32

(52) U.S. Cl. 600/210

(57) ABSTRACT

The present invention is directed to a device for minimizing or preventing damages due to ischemia that can occur within supported or retracted dermal and/or subdermal living tissue, most particularly during surgical procedures, by one or a combination of several means including cyclically applying and reducing supporting or retracting pressure at each of at least two tissue sections into which the supported or retracted tissue is subdivided, bathing these tissue sections with gases and/or liquids such as oxygen and oxygenated blood, presenting low-pressure regions or partial vacuum to areas within these tissue-sections to stimulate bleeding to encourage blood perfusion, controlling the temperature of these tissue sections to forestall ischemic damage, and mechanically moving at least a portion of these tissue sections to stimulate blood perfusion with, for example, a vibrating mechanism.

### BACKGROUND OF THE INVENTION

As surgical retractors provide continuous, unencumbered access to surgical sites, they unavoidably apply pressures to portions of retracted tissue causing tissue compression that restricts perfusion, or the flow of blood. The resultant loss of a continuous supply of fresh oxygenated blood can cause damage to the compressed tissues if perfusion is not restored within reasonable time periods ranging, depending on the tissue types and their locations, from tens of seconds to several minutes or more. Similarly, when areas of a patient's skin are supported or clamped for extended time periods during surgeries, they also unavoidably experience pressures that cause tissue compression which can restrict perfusion. Ischemia, or the condition in which the supply of blood becomes inadequate to maintain tissue vitality, can develop quickly in these compressed areas and tissue damage is the result, leaving the patient with scar tissue or necrosis (tissue death). Unless the surgeon provides for repetitive removal or reduction of pressures that are applied by retraction and positioning devices, there is no available option for preventing this problem. Some brain surgeries require long periods of continuous brain-retraction pressure that can cause loss of function and for many such cases this consequence is considered unavoidable.

Excessive Brain Retraction Pressure (BRP) is said to be the cause of contusion or infarction in 10% of cranial surgery and about 5% in intracranial aneurysms [Andrews R S, Bringas J R. A review of brain retraction and recommendations for minimizing intraoperative brain injury. Neurosurgery 1993; 33:1052-64], while pressure at the retractor blade tip is said to be responsible for 22% of infarctions as determined by CT scans [Rosenorn J. Self-retraining brain retraction pressure during intracranial procedures. Acta Neurochir (Wien) 1987; 87:17-22]

Damage tends to increase as time and pressure increase. Higher pressures produce ischemia at greater depths. Brain tissues are particularly vulnerable, and pressures as low as 10 mmHg (0.193 psi) may impair neurological function [Rosenorn J. and Diemer N., 1985. J Neurosurg 63: 608-11; Yundt K. D. et al., 1997. Neurosurg 40: 442-51].

Muscle injury is closely related to muscle retraction and relaxation during lumbar disc surgery [Kadir Kotil, Tamer Tunckale, Zeynep Tatar, Macit Koldas, Alev Kural, Turgay Bilge, J Neurosurgery—Spine February 2007 Vol 6 Number 2. DOI: 10.3171/spi.2007.6.2.121]. Prolonged use of self-retaining retractors causes reduction in muscle function and is suspected to increase scar tissue generation and postoperative spinal muscle dysfunction [Taylor, Heath; H. McGregor, Alison; Medhi-Zadeh, Siroos; Richards, Simon; Kahn, Nostrat; Zadeh, Jamshied Alaghband; Hughes, Sean P. F. Spine. 27(24):2758-2762, Dec. 15, 2002].

Instrumented retractors quantitatively related ischemia-onset to applied force in both open- and minimally-invasive laparoscopic surgery [Gregory S. Fischer, Sunipa Saha, Jennifer Horwat, John Yu, Jason M. Zandt†, Michael R. Marohn‡, Mark A. Talamini‡, Russell H. Taylor; Computer Integrated Surgery—ERC, Johns Hopkins University, Baltimore, Md.; †Department of Surgery, George Washington University, Washington D.C.; ‡Department of Surgery, Johns Hopkins Hospital, Baltimore, Md.].

Review of the literature indicates serious interest in the problem but proposed solutions describe only specialized surgical retractors with integrated force and oxygenation sensors that can monitor or report real-time data to the surgeon, and suggest studies to better quantify retraction damage so “safe” thresholds of magnitude and duration can be defined [Ischemia Sensing Organ Retractor, Engineering Research Center for Computer Integrated Surgical Systems and Technology (supported by Core NSF CISST/ERC)]. Such studies can help reduce tissue injury and scar formation but lacking a real solution, patients remain at risk for muscle injury (notably the paravertebral muscles, most particularly in the medial lumbar areas), nerve injury (notably the dorsal ramus, medial branch which innervates the multifidus muscle), infection (SSI, or surgical site infection), and postoperative pain. Risks remain for the surgeon as well since ischemia can extend OR and anesthesia time, expand regions requiring tissue debridement, and increase legal liability.

The CDC recognizes a direct connection between SSI and traumatic tissue dissection and estimates that in 1980, “ . . . an SSI increased a patient's hospital stay by approximately 10 days and cost an additional $2,000 . . . [and that a] 1992 analysis showed that each SSI resulted in 7.3 additional postoperative hospital days, adding $3,152 in extra charges.”; “Excellent surgical technique is widely believed to reduce the risk of SSI . . . [and that] such techniques include . . . appropriately managing the postoperative incision.”; “Mild hypothermia appears to increase incisional SSI risk by causing vasoconstriction, decreased delivery of oxygen to the wound space . . . and subsequent impairment of function of phagocytic leukocytes (i.e., neutrophils) . . . . In animal models, supplemental oxygen administration has been shown to reverse the dysfunction of phagocytes in fresh incisions. In recent human experiments, controlled local heating of incisions with an electrically powered bandage has been shown to improve tissue oxygenation.” [US Department of Health and Human Services: INFECTION CONTROL AND HOSPITAL EPIDEMIOLOGY April 1999, Page 254, 263, and 263 respectively, Guideline for Prevention of Surgical Site Infection, 1999, Hospital Infections Program, National Center for Infectious Diseases, Centers for Disease Control and Prevention, Public Health Service, and internal references]. The interesting references to hypothermia and oxygen administration become important findings as they relate to characteristics of the present invention, mentioned later in this application.

Adhesions comprise another area of concern but their relation to surgical retractors is uncertain. Still, compressed tissue, denied the opportunity to remain moist, invites speculation into the potential benefit of providing lubrication to retracted tissue, especially when one reads an advertisement for Sepracoat, a commercially available covering to protect tissues during surgery —“Sepracoat is applied to tissues intra-operatively at the very beginning and throughout the surgical procedure to provide a hydrophilic protective barrier to tissues during the surgical process . . . to reduce the amount of tissue damage that can occur from desiccation or manipulative abrasion. What it is doing is maintaining and perhaps enhancing, during the surgical procedure, the natural tendency of the tissue to be lubricous and not stick together. It therefore reduces what we call de novo adhesion development.”[FOOD AND DRUG ADMINISTRATION, CENTER FOR DEVICES AND RADIOLOGICAL HEALTH, GENERAL PLASTIC SURGERY DEVICES; Office of Device Evaluation, 9200 Corporate Boulevard, Room 20B, Rockville, Md. Proceedings By: CASET Associates, Ltd., 10201 Lee Highway, Suite 160, Fairfax, Va. 22030. (Open) PANEL MEETING—May 5, 1997]. The interesting reference to tissue lubricity also becomes an important finding as it relate to characteristics of the present invention, mentioned later in this application. Also interesting is the seriousness of adhesion related disorder (ARD), a condition accompanied by crippling pain, often misdiagnosed due to its invisibility on standard medical tests, with surgery reported to be its leading cause [Doctors: Bound By Secrecy? Victims: Bound By Pain!, E.L.M. Publishing, Inc.; 1st edition (2007), ISBN-10:0978698207, ISBN-13: 978-0978698201].

Other references make associations between retractor use and tissue damage. For example, “External compression by a retractor increases the intramuscular pressure and decreases local muscle blood flow . . . . Metabolic changes and microvascular abnormalities occur . . . . A pathogenic mechanism for the muscle injury is based on compression and ischemia of the affected muscle. Two hours of continuous retraction caused significant histologic changes and neurogenic damage including degeneration of the neuromuscular junction and atrophy of the muscle. In an animal model, muscle injury after surgery was related to the retraction time and the pressure load generated by the retractor . . . muscle injury after posterior surgery might cause postoperative low back pain and compromise the functional integrity of the muscle . . . . The medial branch of the dorsal primary ramus . . . innervates the multifidus . . . . This dorsal (posterior) ramus is damaged by posterior lumbar procedures.” [Screws, Cages, or Both?—Rick C. Sasso, M.D., SpineUniverse.com http://www.spineuniverse.com/displayarticle.php/article1363.html]

Another states, “The dissection required for internal fixation placement and the significant muscle compression generated by fixed retractor systems utilized in posterolateral fusion procedures with pedicle screw fixation has been shown by histological study and EMG to cause areas of permanent muscle dysfunction and fibrosis described as ‘fusion disease’.” [Failed back surgery syndrome, Martin A. Nogues, Historical note and nomenclature. http://www.medlink.com/medlinkcontent.asp]

In a study measuring mechanical properties of soft tissues to determine breaking points of different organs, the finding most relevant to the present invention was this: “Applying a minimal retraction force causes a significant drop in the local tissue oxygen saturation.” More specifically, the authors found that “Repeated extension of tissue to a fixed position requires decreasing force. During the extension of a tissue sample, the force first raises to a maximum . . . . Then, the extension force drops, though the sample is further stretched. Macroscopically, the extended tissue seems to be intact at this first tear point. Histological examination on the other hand shows real tissue damage with bleeding. Every tear-point on the curve corresponds with a supplementary histological damage. The last tear-point on the curve corresponds with the complete rupture of the tissue . . . . Results after repeated extension suggest microscopic trauma or functional alterations of the tissue after extension.” [G. De Win, B. Van Cleynenbreugel, G. De Gersem, M. Miserez, D. De Ridder, J. Vander Sloten KULeuven, MECHANICAL PROPERTIES OF SOFT TISSUE IN EXTENSION, METHODOLOGY AND PRELIMINARY RESULTS, Serum Creatine Phosphokinase Activity and Histological Changes of the Multifidus Muscle: A Comparative Study of Discectomy with or without Retraction—World Spine Journal, worldspine.org/Documents/WSJ/proceedings/wed_{—}1_disc_surg.pdf and internal references, Belgium Poster B14].

Clearly, due to the publication of these and other studies, surgeons are not oblivious to this problem. As mentioned, several approaches have been proposed to reduce tissue damage caused by surgical retraction, but these have presented, through a variety of approaches, solutions that are to varying degrees more cumbersome, less effective, and/or narrower in their ranges of application than the comprehensive solution provided by the present invention, as described here. In particular, several disclosures essentially provide the surgeon with a measurement of applied pressure using means described, for example, in U.S. Pat. Nos. 3,888,117, 4,263,900, 5,201,325, 5,769,781, 7,325,458, and U.S. Patent Application Publication No. 2006/0025656. Other teachings provide the surgeon with a means of monitoring or being alerted by an annunciating signal initiated by measurements of systemic parameters related to the physiology of the patient and/or the compressed tissue, and/or parameters associated with the physical retractor using means such as those described in U.S. Pat. Nos. 4,784,150, and 4,945,896. Some disclosures similarly incorporate such measurements but add the capability of automatically adjusting, releasing, or equalizing the surface retraction pressure as in U.S. Pat. Nos. 4,263,900, 5,201,325, 6,730,021, and U.S. Patent Application Publication No. 2007/0276188. In U.S. Patent Application Publication No. 2007/0287889 a means of cushioning the surface of a retractor is disclosed for use in minimally invasive, or single-port-entry procedures, including those that are robotically assisted. Similar cushioning approaches applied to non-minimally-invasive surgeries are taught in German Patent Applications Numbers DE29718163 and DE20001813. U.S. Pat. No. 6,733,442 discloses a retractor having a thermal transfer region for cooling retracted tissue, creating an effect that is opposite to the finding of a study mentioned elsewhere in this application suggesting that maintaining tissue warmth is more beneficial than allowing tissues to cool below normal body temperatures. The severity of problems created by brain retractors is addressed in U.S. Pat. No. 7,153,279 by disclosing a device that cushions the rigid edges of a brain retractor. For any benefit to be realized by the surgeon and the patient, the majority of these offer well-intentioned solutions for which the surgeon must interrupt the surgical procedure and take action to realize benefit. The consequences of such interruptions, however, increase surgery time, risks, and costs.

A “Surgical retractor apparatus and method of use” described in abandoned U.S. Patent Application Publication No. 2002/0022770 offers a solution comprising a plurality of inflatable chambers interposed between the blade of a surgical retractor and the retracted tissue to avoid prolonged, static application of pressure to any particular portion of the retracted tissue. These inflatable chambers are to be sequentially inflated and deflated and, in so doing, perform one of the basic functions of one of the embodiments described herein, therefore most closely emulating an actual solution to the problem of retractor-caused ischemia, muscle fiber injury, and nerve damage inherent in present retraction-requiring surgical procedures. An important difference between the present invention and this abandoned application is acknowledgement and discussion of problems controllable only by strict regulation of fluid volume and pressure, and the sizes and shapes to which the inflatable chambers must be constrained to reduce the potential for ruptures and avoid losses of retraction pressure in regions in which it is desired.

To address the need for, and to provide benefits of a system that could eliminate or retard the development of tissue damage in retracted and supported and/or clamped tissues without causing interruption of surgical procedures, the present invention was developed to provide surgeons with a means for reducing or removing such pressures during appropriate intervals.

Notable is the latter purpose addressed by this invention; namely to eliminate or retard the development of tissue damage in areas that are supported and clamped in preferred positions since such purpose primarily applies to a device that heretofore has not existed and is therefore both novel and unique. Specifically, that application toward which the present invention is directed, namely for maintaining tissue health in positioning and clamping situations, is head-clamping, a function that is important in spine surgeries where neck traction is required, and critically important in brain surgeries requiring stable correlations to MRI and X-ray images, respectively. Not offering the advantages provided by the head clamping aspect of the present invention are various means of supporting the head during brain and other cephalic surgeries involving either pins that ensure registration of the head to correlated images, or pads that tightly clamp regions of a patient's head, or both. For example, U.S. Pat. No. 4,169,478 shows a “crown of thorns” head clamp, often referred to as the Mayfield head clamp as illustrated in a drawing within the present application, with which the skull is rigidly held between three skin-piercing and skull-piercing pins. Examples of others that also incorporate pins, pads, or both pins and pads include U.S. Pat. Nos. 2,452,816, 3,099,441, 3,835,861, 4,169,478, 6,306,146, 6,315,783, 7,117,551, and Italian Patent No. 478,651. By contrast to the advantages of the present invention's atraumatic benefits, the disadvantages of the present systems are revealed in both the literature and in documents accessible from websites within the United States government. For example, in just one Newsletter of the Food and Drug Administration, #19 Dec. 2007, patients are reported to have developed scalp lacerations as long as five to six inches and a skull fracture from Mayfield products' skull pins that have moved or slipped, or have been stuck due to an inoperative release mechanism. Locking system failure caused head-slippage from the pins in one case and swivel adaptors had slipped in another. A further problem with head-pinning is the lack of the understanding by surgeons and residents that is necessary to estimate the magnitudes and directions of resultant forces, or force vectors created by energetic use of the surgical instruments with respect to the areas to which these forces are applied; indeed, this inventor personally witnessed patients' heads becoming dislodged from Mayfield head clamps during exposures by one senior surgeon vigorously scraping their skulls during two different surgeries. The potential value of this technology, therefore, in both retraction and head-clamping applications, is considered to be of high value.

This method of providing periods of pressure-relief was anticipated to be effective after reviewing the literature on tissue-damage causes and characteristics; for example, one study¹ of retractor effects found retraction rest periods to be correlated with improvements in postoperative pain, serum CPK, and histological data. This method was subsequently conceived and pursued by the present three inventors and disclosed in the United States Patent Office Provisional patent application titled, “Massaging Retractor” filed 09-07-2007, No. 60/967,646, and further disclosed in the United States Patent Office Provisional patent application titled, “Perfusion Stimulating Retractor” filed 01-28-2008, No. 61/062,482.

On this principle, then, when constriction of blood capillaries is interrupted for an acceptably short time, blood perfusion is partially or fully restored shortly after retraction pressures and support/clamping pressures are removed. The cyclic application and reduction, or cyclic application and removal of pressure enable sufficient perfusion to be maintained over the course of the surgical procedure to enable uninterrupted continuance. At all times during surgeries, through this repetitive process, a sufficiently large portion of tissue surface-area(s) receive(s) pressure sufficient to safely hold-open access openings, or wounds, and/or maintain head and other body-section positions, maintaining tissue vitality through intermittent or continuous perfusion-restoring processes that can be invisible to the surgeon and the assisting staff. Any acceptable pattern of pressure-application zones and any number of operating states may be used. For example, one model of the Perfusion Stimulating Retractor, operating on this principle, could follow a repeating two-state pattern during which, for each repeating cycle, pressure is reduced or removed for a one-minute period from one region or a set of specific regions that constitutes approximately half of the entire area adjacent to and within the footprint of this retractor, after which pressure is then reinstated to this first region just before, or while pressure is reduced or removed for a similar time-period from the remainder of this entire area adjacent to and within the footprint of this retractor. As a further example, a second model of this Perfusion Stimulating Retractor could follow a repeating three-state pattern during which, for each repeating cycle, pressure is reduced or removed for a one-minute period from one region or a set of specific regions that constitute(s) approximately one-third of the entire area adjacent to and within the footprint of this retractor, after which pressure is then reinstated to the first region(s) just before, or while pressure is reduced or removed for a similar time period from a second region or a set of specific regions that constitute(s) approximately a second one-third of this entire area adjacent to and within the footprint of this retractor, after which pressure is then reinstated to the second region or set of specific regions just before, or while pressure is reduced or removed for a similar time period from a third region or a set of specific regions that constitutes approximately a third one-third of this entire area adjacent to and within the footprint of this retractor. In this second example, as could also be true for four-state and higher-number-state Perfusion Stimulating Retractors or similarly operating supporting and/or clamping devices, preferential sequencing of the regions or sets of regions could cause the flow of blood in the retracted tissues to generally travel in specific directions where, for example, stimulating perfusion in the direction(s) in which normal blood flow would occur, would be desirable. For simplicity, applicable drawings and explanations within this application reflect, at most, a three-stage pressure-reduction cycle.

Retractors and support/clamping devices that produce such pressure-shifting may be designed to have any type and pattern of elements or components. They may be driven to have any desirable transition rates, including very slow transition rates that allow pressures to be gently applied by one surface or set of surfaces after, during, or before gently decreasing pressure at another surface or set of surfaces. As an example, a perfusion-stimulating retractor of any type described herein may have parallel elements that move toward and away from retracted tissue areas with respect to interleaved parallel elements. As a second example, a self-retaining Perfusion Stimulating Retractor, similar in appearance to the Weitlaner Self-Retaining Retractor, may have two sets of retraction fingers on each side, each supported by a separate supporting arm, such that one set of retraction fingers can be nested between the retraction fingers of the other, moved independently, and locked into position, allowing retraction pressures to be quickly shifted from one set of retraction fingers to the other set of retraction fingers. As a third example, hydraulically and pneumatically actuated expansion-limited inflatable arrays having separate balloon-like elements held in fixed positions, or molded sections comprising expansion-limited inflatable cavities, may be attached to existing retractor blades to provide inexpensive, single-use alternatives to reusable but more expensive models. As a fourth example, perfusion-stimulating retractors incorporating one or more sets of rollers in continuous or intermittent motion can supply massaging-like action, bidirectionally or unidirectionally, the latter which can encourage blood flow within the surface of the retracted tissue in preferential directions. In one simple configuration for this example, two parallel-mounted sets of rollers move toward and away from each other to eliminate the lateral forces that would be created by movement of a single roller-set during use.

Other influences, such as exposing tissues to higher concentrations of oxygen, or continually wetting their surfaces with, for example, oxygenated blood or a blood-thinning agent such as Heparin, could help to retard or prevent injury to retracted tissues. For example, lung transplant operations tolerate longer transition periods between lung-harvesting and implantation when donor tissue is kept in highly oxygenated solutions [BBC, “XVIVO Lung Perfusion System” with bloodless solution containing oxygen, proteins and nutrients keep lungs stable ex vivo allowing repair, Toronto General Hospital http://news.bbc.co.uk/2/hi/health/7791252.stm], suggesting that bathing retracted tissues with oxygen, oxygenated blood, or both, supplied through small openings in the surfaces of the retractor's movable or inflatable segments, could prove beneficial. Temperature is another known influence, and with some surprise, it has been shown that tissue health is extended when kept warm rather than being cooled by cool ambient air or by heat-sinking by cold retractor blades, so providing retracting surfaces that are warmed could also prove beneficial. Other influences, such as a partial vacuum applied to sections of retracted tissue surfaces, or perforated retraction areas that present low-pressure zones to encourage slow and continuous bleeding at the tissue surfaces, are more theoretical and must be studied to determine the degree to which perfusion in retracted tissues can be stimulated.

Clearly, there are multiple device-configurations of perfusion-stimulating retractors that can employ this principle, as well as potentially enhancing influences that could enhance their efficacy. As a result, it would be tedious and perhaps even confusing to identify all of the possible implementations that could be made using combinations of the “variables” available for implementing models for particular applications. Better it is to identify these variables, and then list the most logical models that could be developed after choosing specific combinations that best meet the needs of the commonest applications. A list of these variables appears in the Detailed Description of the present application.
^{1H}Journal of Neurosurgery, February 2007 Volume 6, Number 2; DOI: 10.3171/spi.2007.6.2.121, Serum creatine phosphokinase activity and histological changes in the multifidus muscle: a prospective randomized controlled comparative study of discectomy with or without retraction

### SUMMARY OF THE INVENTION

Studies confirm that cyclical removal of surgical retraction pressure can reduce or eliminate ischemia, or lack of blood perfusion, in retracted tissues. Exercising this option with conventional retraction systems significantly increases cost and risk, however, and few if any surgeons employ this technique. To provide a commensurate benefit while maintaining uninterrupted access to the surgical site, the retractors described herein have been designed to controllably apply and reduce retraction pressure at each of a number of tissue sections into which the retracted tissue is subdivided. Each retractor, along with its automatic or manual controlling and driving means, comprises a subset of a system, using this single method, whereby it can operate like two (or more) retractors in one. Smaller models employed in cephalic surgeries can preserve brain tissue and brain function, while larger models prevent tissue injury, and potential necrosis, over a wide range of surgeries. Using this same method, models used on external tissues can preserve the vitality of regional skin and subdermal tissues while simultaneously providing surgery-facilitating supporting and clamping functions.

Designs include mechanical and fluid-driven configurations that are either stand-alone devices, or assemblies that attach to either common retractor blades or to body-region-support and/or body-region-clamping hardware, such as head clamps. Fluid-driven units can operate automatically and include designs for minimally invasive procedures. Some mechanical devices can be manually operated, and variations of these devices include a Weitlaner-like (self-retaining) retractor, while others can operate automatically.

Additional variations of this system include value-added characteristics having the potential of contributing to patient safety. One example of potential added-value includes a three-state design that can direct stimulated perfusion in preferential directions. Others include surface perforations for bathing tissue surfaces with oxygen, oxygenated blood, blood-thinning agents, or other fluids; similar perforations for tissue communication to ambient air or partial vacuum to encourage localized bleeding and therefore blood-movement within the tissue; surface-temperature control; and/or vibrating/massaging influences that can be applied to the tissues.

A primary design-focus of the present invention has been continuous recognition that all models must meet requirements of the United States Food and Drug Administration, the Joint Committee on Accreditation of Healthcare Organizations (JHACO), and a typical hospital Internal Review Board for devices that are to be used in the operating room.

### BRIEF DESCRIPTION OF THE DRAWINGS

The accompanying drawings incorporated in and forming a part of the specification illustrate several aspects of the present invention and, together with the description and claims, serve to explain the principles of the invention. In the drawings—

FIG. 1 provides a view of each side of one possible design of a single element, or “finger” of a mechanical atraumatic retractor. Depending on the design and application, all such elements used in an associated mechanical retractor may have identical characteristics, or each single element may have a specific thickness, height, curvature profile, and means of attachment to its supporting structure with, such as for example, a hook on its upper surface (not shown), adhesive, or weld. The concave “inside” surface, revealed on the right-hand side of right-hand retractor element 11 is the surface presented to the tissue to be retracted in surgical procedures.

FIG. 2 is a drawing showing, for illustrative purposes, the retractor element 11 of FIG. 1 mounted on a supporting bar 13 that, with all neighboring supporting bars, together comprise a platform, within a plane, of separate components that have freedom of motion, within a range, either in one direction along parallel paths, or the exact opposite direction. In this example, eight such supporting bars are grouped such that all even-numbered bars, as numbered from the nearest-appearing side, are linked to move together as a unit, or set, and all odd-numbered bars are similarly linked to move together as a set. A mechanism, automatically or manually driven, is integrated with these components to move the odd-numbered set of bars, and thereby also their odd-numbered retractor fingers of which finger 11 is one, toward the retracted tissue, thusly applying retraction forces at their concave surfaces, or alternatively, away from the retracted tissue, withdrawing retraction forces and thereby transferring retraction forces to the even-numbered fingers. In an alternate configuration, this or a similar mechanism is integrated with these components to simultaneously move the even-numbered set of bars, and thereby also their even-numbered fingers, of which finger 12 is one, in the direction opposite the direction of motion of the odd-numbered fingers. For ease of reference, the entire assembly of supporting bars, their linkages, and any necessary levers, cams, supports, bearings, and housings are referenced herein as a slider mechanism.

FIG. 3 is a drawing to illustrate one way to achieve linked motion of the similarly odd-numbered supporting bars of FIG. 2. Alternate supporting bars, of which supporting bar 16 is one, are shown with fixed extensions that are linked by a linkage arm 15 at pivot points, the uses of which help to equalize retraction forces at the concave forward faces of the associated retractor fingers. A solenoid 14 is shown with its plunger attached to a center-of-forces pivot-point illustrating one means whereby motive force can be applied to this set of supporting bars, thereby causing this two-state retractor to transition from one state to the other. In this drawing, the remaining alternating supporting bars, of which supporting bar 17 is one, remain in a fixed position relative to the appropriate housing mentioned above.

FIG. 4 is a drawing showing an exemplary lower-surface-view of the supporting bars of FIGS. 2 and 3 revealing slots that can accept and secure the positions of their associated retraction fingers.

FIG. 5 is a drawing showing the supporting bars of FIG. 3 with a set of linkage arms 19 for one set of alternating support bars, and a set of linkage arms 20 for the other set of alternating support bars. In these configurations, as a consequence of the motional freedom at the pivot points, each linkage-set applies forces to its associated supporting-bar members in such a way that their associated retractor fingers apply equal retraction forces to the retracted tissue, irrespective of the travel distances of the supporting bars, provided that their excursions are within their limited ranges of motion.

FIG. 6 is a drawing showing the supporting bars of FIG. 3 with linkage sets 19 and 20 linked by a lever having pivot points at its ends for attachment to separate solenoids 21 and 22. As with the mechanism of FIG. 5, force equalization is achieved with each of the linkage sets. The distance differential between the retractor fingers is doubled, as compared to the arrangement shown in FIG. 3, as transition is made from one state to the other. The state-change occurs when the activated solenoid is deactivated and the other solenoid is activated.

FIG. 7 shows the components of FIG. 6 with the addition of a means for changing the states of articulated retraction-finger claws (not shown), if such claws are provided and state-change is desired. To provide the additional means of controlling such claws, component 25 is shown to represent either a rigid rod driving a rocking lever 26 which in-turn drives a rigid rod (not shown) extending into the page toward, and linking to, a claw-articulating mechanism (also not shown), or, a flexible cord that extends toward and part-way around component 26, for this arrangement a pulley, finally causing a state-change of said articulating claws.

FIG. 8 shows, for illustrative purposes, an exemplary assemblage of retractor fingers and supporting bars assembled in a thin housing (with driving-mechanism components not shown) attached to a support arm that could be anchored to retractor-support hardware, such as the ring of a Bookwalter Retractor Set. The second drawing of FIG. 8 shows the retractor fingers with a larger scale.

FIG. 9 is a drawing of the assemblage of FIG. 8, an exemplary two-state retractor, showing a representative set of stages as it transitions from one state to the other.

FIG. 10 depicts a set of retraction fingers that could be configured as support bars with integrated retraction fingers.

FIG. 11 shows a sectional side-view of a retractor model comprised of a set of parallel helically-shaped flexible rods. Its nearest-appearing helical rod 35 is shown sectioned axially in the plane of the paper along with a sectional top view of the set of rotating helical components, shown as the top-most row of nine such views, at a fixed position 36 and at a fixed point in time. Use is made of the fact that parallel arrangements of alternately-90-degree-offset rotating helical rods (36 and 37) having specific helical-shapes will “nest” everywhere along their lengths when the alternating helical are rotated in one direction and their interstitial neighbors are rotated in the opposite direction at the same rotation rate, a condition ensured by a set of equally sized gears, like the gear 38 on the first helical, affixed to the bottoms of the rods. Preferably with an elastic isolation sheet separating the rotating rods from retracted tissue, in a way similar to the isolation provided by the outer upholstery material of a back-massaging chair, the peaks and troughs of appropriately-sized rotating rods will present the maximum-to-near-minimum range of retraction forces and indentation distances that are optimum for tissues ranging from brain tissue to muscle tissue. A helical-element retractor can be operated continuously, acting as an infinite-state, ever-changing retractor (essentially operating as a massaging device), or made to have selected two-, three-, or other multi-state (such as 90-degree) transitions at desired intervals.

FIG. 12 is a drawing showing top, rear, and side views of an exemplary nested-helical mechanical retractor. It should be noted that with flexible rods axially positioned at each end and with elastic-surface support over part or all of its rear surface, the retractor's front surface can be made to conform, to a limited degree, to surfaces that are not only curved about one axis but are effectively concave or convex. Rotational driving force can be supplied using a thin, speedometer-type cable 39 with its outer sheath attached to the helical retractor housing. Further, although usable as a body-section support device, it would be counterproductive in body-section clamping situations since the supported tissue could have a tendency to “walk” as the helical rods rotate.

FIG. 13 is a drawing of a sliding-rack retractor that provides a massaging action similar to that of the nested-helical retractor of FIG. 12 in that it has a sliding component 43, with sections 45 in raised-relief, that is made to move, through force applied to slider extension 44, between a fixed surface 48, shown with the shape of a conventional retractor blade, and an elastic isolation sheet 46 which together form a kind of pocket, further into which and partially out of which this sliding component is made to move through manual or automatically initiated action.

FIG. 14 depicts a fully assembled exemplary retractor of the type shown in FIG. 13.

FIG. 15 shows a close-up view of a section of a smaller-model sliding-rack retractor in which the shown rack is a more suitable rack-component for brain-retractor applications.

FIG. 16 is a drawing of the sliding-rack retractor of FIG. 14 configured for manual operation and attachment to retractor-mounting hardware. Manual operation is initiated through rotation of a knob 51 which drives a cam 53 that rides in a slot in a slider extension 44 configured for this retractor model which is equipped with mounting holes 52 for attachment to a suitable retractor support arm.

FIG. 17 shows one example of a modification that can be made to the sliding-rack retractor of FIG. 16 to allow it to be actuated by remote control. Substituting for the knob 51 that is used to drive the cam 53, a mechanical assembly 57 provides the necessary rotational motion to drive the cam with power and control supplied through an umbilical 56 consisting of, for example, electrical wires carrying power to drive a motor or solenoid, a sheathed cable like a speedometer cable that powers this actuation with rotational and/or translational movement of its inner wire, or flexible tubing that conducts fluid to a cylinder or fluid motor. For several of these, of course, actuation of the slider extension 44 could be accomplished directly without the need for a cam and slot.

FIG. 18 depicts a modification of the sliding-rack retractor of FIG. 17 whereby additional components interconnect with movable teeth hinged and mounted in the bottom area of the retractor assembly.

FIG. 19 acknowledges the need for any suitable driving unit that can facilitate remote control and operation of any of the tissue-positioning devices that operate on the principle of pressure-interruption to preserve tissue health. As earlier implied, for many of these models, its output may be mechanical motion, motion of fluid, or electrical, with control supplied by timer, microprocessor, computer, or the like.

FIG. 20 is a drawing of the base plate 65 of a mechanical tissue-positioning device, shown here as one of the set of flexible components of a retractor identified as a flexible grate retractor. As described more fully in the detailed description, the base plate is constructed with diagonal slots 66 that guide movable fingers to apply lifting forces to one of two grate-resembling groups of parallel segments.

The drawing on the left side of FIG. 21 illustrates a front- and right-side-view of segments 67 held together by symmetrical semi-rigid straps 68 which together form one of the grate-like groups of segments (or simply one of the grates) of this flexible-grate retractor which, in this configuration, remains affixed to the base plate 65. The drawing on the right side of FIG. 21 shows a front- and right-side-view of these two components after they are joined together.

FIG. 22 illustrates a front- and right-side-view of identical segments held together by symmetrical and farther-separated semi-rigid straps 69 which together form the second of the grate-like groups of this flexible-grate retractor which, in this configuration, can move toward and away from the base plate in trajectories guided by the segments of the fixed-position grate shown in FIG. 21.

The position of the movable grate in its normal operating position is shown in the drawing of FIG. 23.

The movable fingers 71 that apply lifting forces to the movable grate are individual position-restorable springy and flexible tabs, formed in this example from a single die-cut sheet to resemble the drawing of FIG. 24.

A close-up view of a section of the resulting finger sheet is shown in FIG. 25.

FIG. 26 shows the placement of the finger sheet in the flexible-grate retractor (without any of a variety of functional guides that could maintain its position).

FIG. 27 illustrates the influence of the finger sheet as it is moved to the left in the drawing causing its fingers to travel into the diagonal slots and eventually push the movable grate upward.

FIG. 28 shows a set of seven rollers 75 along with a companion set of seven rollers that can present retraction-pressure regions along the nearest-seeming surfaces. In operation the sets of rollers are driven to move toward each other, (preferably by a sheathed cable that draws the sets together) synchronously, due to end-mounted gears 76 that ride on a rack 77 while being held in constant relative positions by frame sections 78 ensuring equal travel rates and distances, and then away from each other, in a cyclic fashion, causing the original retraction-pressure regions to be swapped to similar-sized regions when the roller sets are adjacent to each other.

FIG. 29 illustrates how pairs of cylinders 81 attached to gears 82 that cause them to be alternately moved clockwise and counterclockwise about their common axes when any pair are so-driven, can present alternating retraction-pressure surfaces along the surface of a flexible isolating membrane 80.

FIGS. 30 through 35 are cross-section views of nine sections of tubing that can be disposed between retracted or other tissue and an existing retractor blade or other fixed surface to achieve changes in position of pressure applied to the tissue brought-about by variations in fluid pressure to which the sections of tubing are subjected. These drawings also show the expansion-limiting influence of an inelastic covering 87 and the profile that would be assumed by retracted tissue when presented with an atraumatic retractor of this type having a flexible membrane covering the surfaces of the retractor segments applying retraction pressure.

FIG. 36 shows an example of an atraumatic retractor prototype that was constructed using inelastic tubing bonded to the convex surface of an existing retractor blade 95. Alternate tubing sections 96 are shown deflated while alternate tubing sections 97 are shown inflated.

As an example of an extrusion of parallel tubes, FIG. 37 illustrates the general appearance of an atraumatic retractor formed from it when half of the sections are deflated.

FIG. 38 illustrates how positions of applied pressure may be swapped when inflation and deflation cycles are imposed upon only a single group of alternating inflatable segments. This is made possible by using fixed, midpoint-position segments at all alternate positions.

FIG. 39 shows an example of an atraumatic retractor constructed using inelastic tubing for the retractor segments in an arrangement of components that allow it to be used with a common Kelley retractor blade. Operation of this type of retractor is discussed in great detail in the next section.

FIG. 40 is a rear-view of a similar configuration using components that are differently interconnected and considered more amenable to fabrication using molded components.

FIG. 41 is a similarly constructed fluid-operated retractor with an added function provided by perforations 120 in the forward-facing surfaces of the inflatable chambers. These perforations enable the retracted tissue to be bathed with liquids and gases such as oxygen and oxygenated blood while simultaneously providing retraction pressures with their presence.

FIG. 42 illustrates the profile of retracted tissue 123 when a three-state atraumatic retractor is used. Four positions of reduced retraction pressure 124 are shown.

FIG. 43 uses an elastic partial-covering of a fluid-operated retractor to enable it to be held to a retractor blade in much the same way that a fitted sheet is held to a mattress.

FIGS. 44 through FIG. 46 relate to an extrusion 130 that can be cut-to-size to serve as an array of inflatable retractor sections that could fit a variety of retractor blades while providing adjustable-width sections for application flexibility.

FIG. 47 illustrates a method of creating nested inflatable sections 144 using stacked components that could be made from molding and/or vacuum-forming processes.

FIG. 48 is a representation of fluid-handling and control components that may be incorporated into a single enclosure to provide fully automatic control of fluid-operated tissue-positioning and retraction devices.

FIG. 49 is a drawing of a three-section two-state fluid-operated atraumatic retractor showing both sides of all three sections. This model also is amenable to fabrication at least partially employing a molding and/or a vacuum-forming process.

FIG. 50 shows the component-assembly order for the retractor of FIG. 49.

Front-views of the assembled retractor of FIG. 50 are depicted in FIG. 51 to show its appearances when all segments of the retractor's segments are inflated and when one group of the retractor's segments are deflated.

FIG. 52 shows one component of a three-state retractor built using the principles of the immediately previous retractors.

FIGS. 53 and 54 show the assembly order and the complete assembly, respectively, of a three-state similarly fabricated Atraumatic surgical retraction and head-clamping device that may be used as a substitute for the skull-piercing pins of the Mayfield Head Clamp shown in FIG. 77.

FIG. 55 shows the rear component of a device similar to the atraumatic surgical retraction and head-clamping device of FIG. 54 but modified for use as a three-state atraumatic retractor where, as in an earlier example, the device incorporates a pocket within a flexible layer that can accept a blade for easy attachment. FIGS. 56 through 59 show the additional layers of this retractor shown individually, and FIG. 60 shows the completed assembly.

FIGS. 61 through 68 show component views and assembled-mechanism views of a mechanical flexible-grate atraumatic retractor constructed to be specifically appropriate for use as a brain retractor.

FIG. 69 shows front- and side-views of a nested-helix retractor constructed to be specifically appropriate for use as a brain retractor.

FIG. 70 illustrates the operating principle of a sliding-rack retractor that operates like the retractor of FIG. 13 but constructed for specific application as a brain retractor.

FIG. 71 is a multiple-inflatable-chamber retractor usable in conjunction with a conventional brain retractor.

FIG. 72 illustrates how the chambers of the retractor of FIG. 71 might be formed from an extrusion.

FIG. 73 shows front- and side-views of an acoustically-powered brain retractor that supports standing-waves in a fluid-filled waveguide to create low- and high-pressure zones that can be moved by changing the driving oscillator to a different resonant frequency.

FIGS. 74 and 75 are drawings of an atraumatic retractor that can be used for minimally invasive surgeries to hold-open and protect the tissues within the wound.

FIG. 76 illustrates a modification that can be made to a Weitlaner self-retaining retractor that can transform the device into an atraumatic retractor.

FIG. 77 is a copy of a sketch found in the U.S. patent for the Mayfield Head Clamp.

FIG. 78 is a proposed head clamp that obviates the need for skull-piercing pins.

### DETAILED DESCRIPTION OF THE INVENTION

The present invention is directed to a device for minimizing or preventing damage due to ischemia that can occur within supported or retracted dermal and/or subdermal living tissue, most particularly during surgical procedures, by one or a combination of several means including cyclically applying and reducing supporting or retracting pressure at each of at least two tissue sections into which the supported or retracted tissue is subdivided, bathing these tissue sections with oxygen, oxygenated blood, or other gases or liquids, presenting low-pressure regions or a partial vacuum to areas within these tissue-sections to encourage blood perfusion through selective stimulated bleeding, controlling the temperature of these tissue sections to forestall ischemic damage, and mechanically moving at least a portion of these tissue sections to stimulate blood perfusion with, for example, a vibrating mechanism. Although specific embodiments of the invention are here-described with references to the drawings, it should be understood that these embodiments are simply illustrative examples of but a small number of the many possible specific embodiments which can represent applications of the principles of the invention. It should also be understood that the range of possible embodiments employing combinations of these several means is so broad that the more obvious variations incorporating means for vibrating, heating, cooling, and creating low-pressure regions with surface openings or partial vacuum are purposely limited to mention within this application, and that such variations, along with other changes and modifications that may be obvious to one skilled in the art to which the invention pertains, are deemed to be within the spirit, scope, and contemplation of the invention as further defined in the appended claims.

Due to this broad range of possible embodiments, descriptive details in this application are primarily devoted to mechanical and fluid-driven configurations that cyclically apply and reduce supportive or retractive pressure at subdivisions of supported or retracted tissue-regions. More specifically, rather than discussing descriptive details for both tissue-supporting and tissue-retracting applications, this description purposely limits discussion of many aspects of the invention pertaining to the former since they are but a subset of the latter.

In general, this discussion of atraumatic retractor designs is directed toward two-state retractor operation where cyclic reductions and increases in pressure are presented to the tissue by the surface of a structure that is subdivided into either two distinct regions or two sets or groupings of separate segments having arbitrarily shaped areas arranged in any appropriate pattern. A reduction in pressure is produced as a natural result of the surface of one of two distinct regions withdrawing to a position behind the surface of the other of two distinct regions, or by a similar withdrawal to new such inferior positions of the surfaces of one of the two sets or groupings of separate segments. As a consequence, an increase in pressure results as most or all of the retraction load is shifted to the alternate surface or surfaces, as appropriate. Alternatively, an increase in pressure is produced as a natural result of the surface of one of two distinct regions, or the surfaces of one of the two sets or groupings of separate segments, being pushed forward of the surface of the other of two distinct regions, or surfaces of one of the two sets or groupings of separate segments.

Implemented this way, the retractor device can understandably be referred to as a kind of dual retractor that operates like two retractors in one. When implemented to have three or more separate states, a retractor surface can move in what may be understood to be equivalent to a serpentine movement to direct blood perfusion in preferential directions. In any of these implementations, smaller retractor models can function in cephalic surgeries to preserve brain tissue and brain function, while larger models can preserve a wide range of tissues over a wide range of other surgeries. The simple operating principle of the atraumatic retractor in all of these applications is the periodic relief from retraction pressure that it provides, a technique that laboratory studies have shown is effective in preventing ischemia, and its main advantage to the surgeon is that it provides this protection while simultaneously maintaining uninterrupted access to the surgical site.

Atraumatic retractors, as well as their tissue-positioning counterparts, are generally mechanically or fluid-operated. Mechanical devices employ segments comprised of protrusions that have generally forward-facing sides that can be controlled to physically move, individually or in groups, to apply desired levels of pressure to regions of retracted tissues. Fluid-operated devices employ expansion-limited chambers having generally forward-facing surfaces that are made to protrude toward retracted tissues and/or withdraw away from retracted tissues through the introduction of positive or negative fluid pressure. Expansion-limitation of these chambers is achieved either by the use of inelastic materials, or by expansion-limiting sheaths or coverings of fabric or other usable materials. The chambers are typically formed from tubing comprised of (1) materials that render them essentially inelastic, a well-known property of, for example, electrical insulating tubing known as shrink sleeving, (2) from expandable tubing that is contained where necessary by any suitable inelastic materials, woven, solid or otherwise disposed, or (3) from inelastic materials that can seal the openings of cavities in substantially inelastic structures while maintaining the ability to flex and form convex or other ballooned shapes when exposed to fluid pressure sufficient to produce a full range of required retraction pressures added to pressure levels constituting an acceptable safety margin, without rupture or unacceptable weakening from a safe-minimum number of flexions with and without the full range of potential retraction-loading.

To consolidate discussion of many of these variables, we can state that segments or segment surfaces of atraumatic retractors and non-retracting tissue-positioning devices move toward or away from tissues through the application of forces controlled by and/or delivered through any number of mechanical components such as levers, cams, pistons, gears, springs, cables, bellows, and the like, or by the presence of or increases and/or decreases in liquid and/or gas pressure. The ultimate power supplying said forces can be sourced or released by any one of or any combination of human muscle action applied, for example, to knobs, levers, or other protuberances, the application of an increase or decrease in gas and/or liquid pressure, springs or other pre-tensioned devices such as spring-loaded bellows, at least one source of electrical energy, or even ambient air. Regulation of said forces may be accomplished through incorporation of at least one power-mediating device such as a mechanical, electrical, or fluid switch, valve, pump, stopper, cap, or tube-kinking or tube-compressing device, actuation of which may be manual through human interaction with devices listed above, and/or sensing devices, or automatically through intercession by one or more controlling devices such as timers, microprocessors, computers, and the like. In addition, power for actuating the devices may be delivered through at least one of one or more sheathed cables having their axial wires moved rotationally or transversely, one or more flexible tubes, power-conducting materials such as wire, and one or more transducers that convert one form of power to another, such as an electric solenoid or motor. Further, when operating automatically, these controlling devices may be partly or wholly regulated by known or potentially relevant systemic parameters such as blood pressure and expiration gases, or parameters related to proximal tissue such as applied pressure, blood-perfusion, fluoroscopy, histological characteristics, AC impedance, DC resistivity, cell polarization, ionic migration, temperature, thermal conductivity, thermal resistivity, dynamic response to pressure, sonic latency, sonic spectral response, acoustic impedance, reflective spectra, gas absorption, and liquid absorption.

Most mechanical atraumatic retractor designs and some fluid-operated designs are directed toward self-retaining, ring-mounted, stanchion-mounted, or hand-held configurations, whereas various sizes of fluid-driven hydraulic or pneumatic devices are primarily meant to attach to common retractor blades. Collapsible models, designed for minimally invasive procedures, serve to hold-widened a minimally invasive surgical incision or, in some situations, provide widening of the incision as well.

A critical feature of all fluid-driven designs is expansion-limitation of the inflatable chambers. This is preferably accomplished by incorporating fabric or other inelastic composition since the absence of such limitation presents the risk of potential rupture due to ballooning of unloaded regions, a loss of retraction pressure in loaded regions, or both.

Control of the retractors can be separated from the source or sources of power and use alternative means including wireless technology using, for example, RF or photonic (IR, visible, or UV) transmission and reception through air-link or fiber-optic linkage. Manual control is an ever-present alternative, applied directly to the device or applied by remote control of the device or control of the power source.

Organization of these variables, again directed toward the retractor application, helps clarify the range of optional embodiments of this invention. For each relevant aspect, most of the envisioned options, some of which are mentioned only later in this application, are listed below.
Product Stock/Purchase Category—reusable/consumable
Product Deployment—stand-alone/adjunctive (hand-held, ring/stanchion-mounted, insert)
Mechanism Actuation—manual/automatic
Power Source—human (knob, lever), fluid (pressure, vacuum), electric (motor, solenoid)
Power Delivery—flexible tubing, electrical wire, rigid cable (push/pull, rotating)
   Interconnection (permanent/connector-linked, umbilical-length)
Power Control/Regulation—pump(s), valves, timer(s) (mechanical, electric), control circuit (fixed, programmable), sensor(s), gauge(s), location (integrated/remote)
Retraction-pressure Delivery Means
   Mechanical (protruding/withdrawing/sliding/rotating helical segments, etc.)
      Interleaved-fingers, in groups that separately advance/withdraw
      Protruding/retracting Segments, in groups that separately advance/withdraw
   Pressurized Chamber (expansion-limited, expandable and/or collapsible)
      Balloon; Inelastic tubing; Elastic tubing within inelastic casing; Bellows; Cavity Gas-driven; Liquid-driven (controlled-displacement)
   Acoustic Standing-wave
Retractor area—approximately 1 sq. in. (for brain), several square inches (for non-cephalic)
Retractor shape—flat (approx. rectangular for brain), curved (for others and for attachment to blade), circular (for minimally invasive applications)
Segment-shape—long, thin rectangle; hexagonal; circular; square; other
Segment-size—as appropriate
Protective covering—elastic isolation membrane; no covering (as appropriate)
Retractor profile—fixed (rigid), conformable (flexible), adjustable-shape (malleable)
Application—Retractor (brain, other cephalic, non-cephalic); Tissue-positioning (no ins)
Modes —(2-state, 3-state, patterned)
Cycling—duration, duty cycle

The remainder of this section is devoted to the primary functional and operational aspects of the invention as well as some of the specific variations that may facilitate its use and enhance efficacy in specific applications. References are made to drawings to add clarity. Numeral reference designations uniquely identify elements throughout the views.

As already explained, the heart of the invention is subdivision of, and cyclic application and withdrawal of the pressure-applying surfaces of structures that support, position, or retract living tissue during surgical procedures. In its simplest form, subdivisions, or separate sections of these structures are made to physically move toward or away from their proximal tissues frequently enough to maintain blood-flow rates and volumes that are sufficient to maintain tissue vitality.

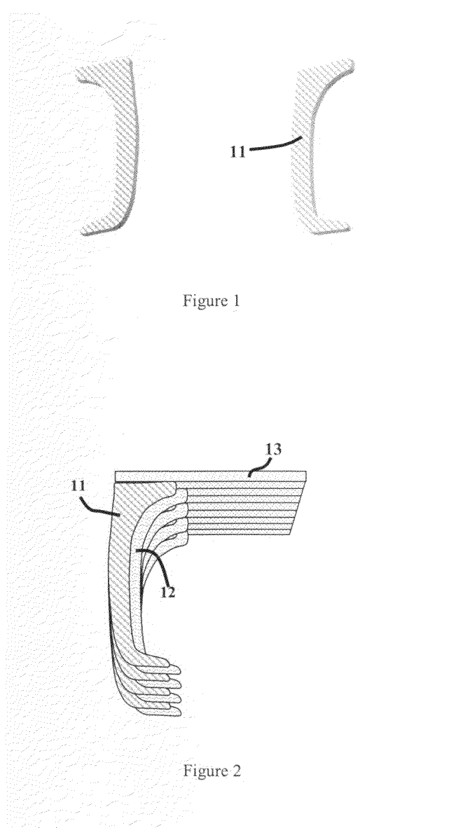

Explained less generically, one can imagine a surgical retractor blade that is cut in half along any path by which each section presents half of the former tissue-contacting surface area. Alternately and cyclically moving each half of the retractor toward and away from the retracted tissue will tend to repetitively impede and then allow resumption of blood flow within it. Imagining further a typical retractor cut along several straight and parallel paths to produce numerous equal-sized segments, the resulting segments might appear like the mechanical retraction finger 11 illustrated in FIG. 1. When a group of such fingers is attached to supports that, at a particular moment of time, move in the same direction and travel the same distances, and similar fingers attached to a different set of supports are either fixed in position or move in opposite directions to the first set, the resulting arrangement could look like the components shown in FIG. 2. In this drawing, the nearest-appearing retractor finger 11 and its coincidentally moving set of fingers is closer to the left side of the page, and its adjacent retractor finger 12 and its coincidentally moving set of fingers is closer to the right side of the page. The concave-like surfaces of these fingers are the surfaces that contact the retracted tissue, so in this drawing, or at this moment in time, finger 12 and its associated fingers are the segments that would be doing the work of retracting the tissue and thereby reducing blood flow in the region of its employ, while finger 11 and its associated fingers would be reducing or removing pressure from the tissue in regions closest to its concave-like surfaces. Movements of the fingers would correspond to movements of their associated supporting bars, all of which would remain in positions parallel to the nearest-appearing supporting bar 13 during resting and transition periods.

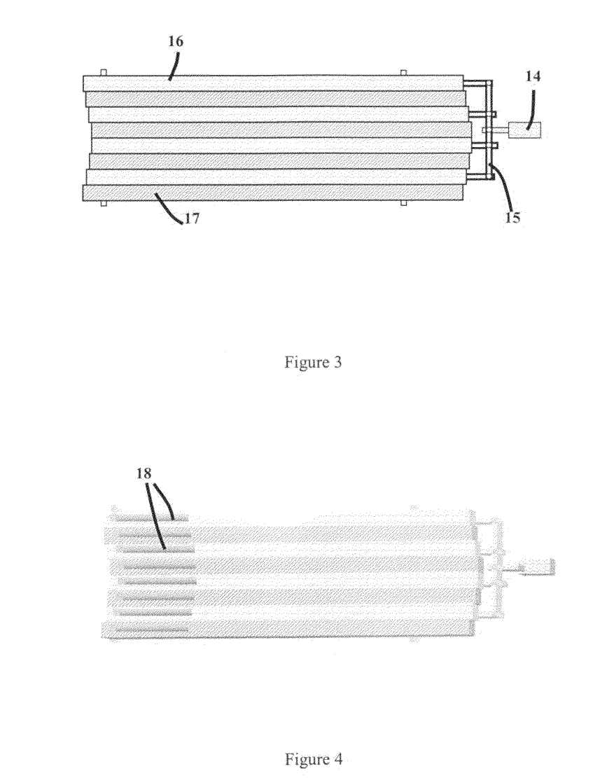

The supporting bars are illustrated more distinctly in FIGS. 3 and 4 where representative supporting bars, which might typically be much shorter than those shown and which might number many more than the eight included in each drawing, reveal surfaces that are opposite the surfaces to which these fingers are held, and opposing surfaces, respectively, having slots 18 that can accept tongues that (with hook formations not shown in these drawings) could project from the upper-shown portions of the fingers and serve as attachment devices. Supporting bar 16 and the other like-cross-hatched supporting bars can be seen linked by crossbar 15, the midpoint of which is connected to the plunger of an electromagnetic solenoid 14 that, when energized, applies tensional forces to these supporting bars which, in turn, can move associated fingers (not shown in these figures) to the right or, for these drawings, toward the retracted tissue, with sufficient force to assume most or all of the retraction pressures, relieving from pressure the areas of tissue that the (also not shown) fingers associated with supporting bar 17 and its like-cross-hatched supporting bars, would otherwise contact.

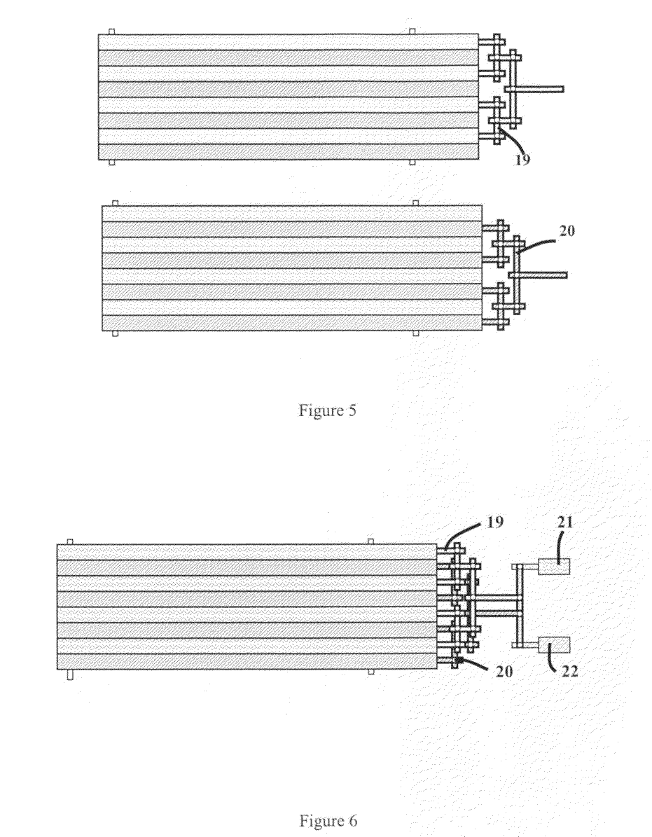

The two drawings of FIG. 5 are meant to display the same set of supporting bars. The top one shows a set of three whippletree-like crossbars 19 connected, by pivot joints, to two interconnecting linkages and the four tongue-like protrusions of their respective, and lighter-shaded supporting bars. The bottom one shows a similar set of three interconnected whippletree crossbars 20 connected to the four tongue-like protrusions of their respective supporting bars. This use of this arrangement of pivoted crossbars serves to equalize the tensional forces applied to the supporting bars, and hence the retractive forces applied by the respective retraction fingers, in a well-known way.

FIG. 6 is a drawing of this same set of supporting bars with both sets of force-equalizing crossbars 19 and 20. Stably mounted solenoids 21 and 22 control the positions of the two sets of supporting bars and hence their respective retracting fingers. Energizing solenoid 21 exclusively drives one set of fingers against the retracted tissue, and energizing solenoid 22 exclusively drives the other set of fingers against the retracted tissue. In both of these states, the fingers supplying retraction pressure will be applied to the tissue with forces that are approximately equal.

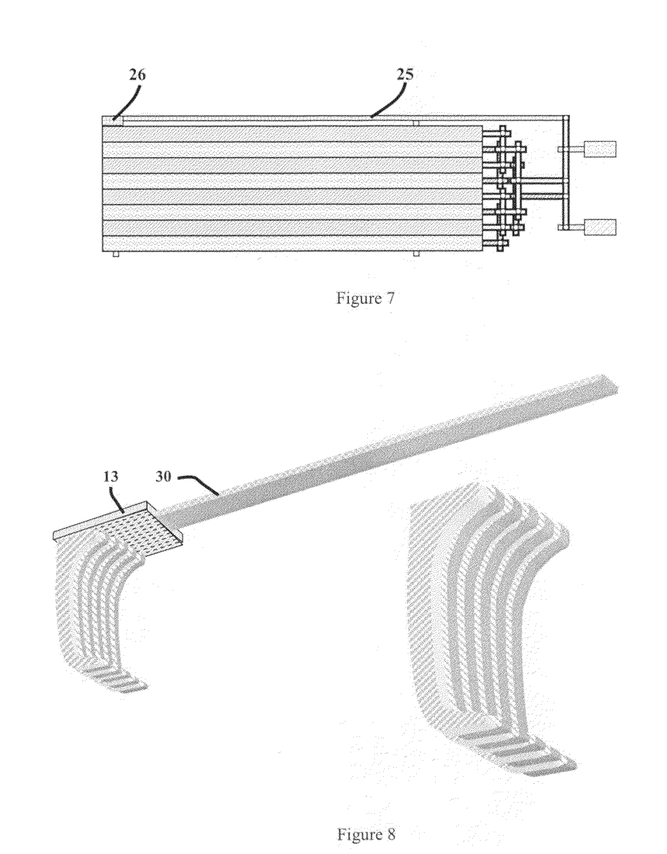

Many retractor blades have relatively sharp teeth along their lower edges to help maintain their positions and prevent dislodgment. In applications where it could be desirable to cyclically present and withdraw these teeth, provision could be made for this using mechanical linkage such as a cable 25 passing over a pulley 26 in FIG. 7.

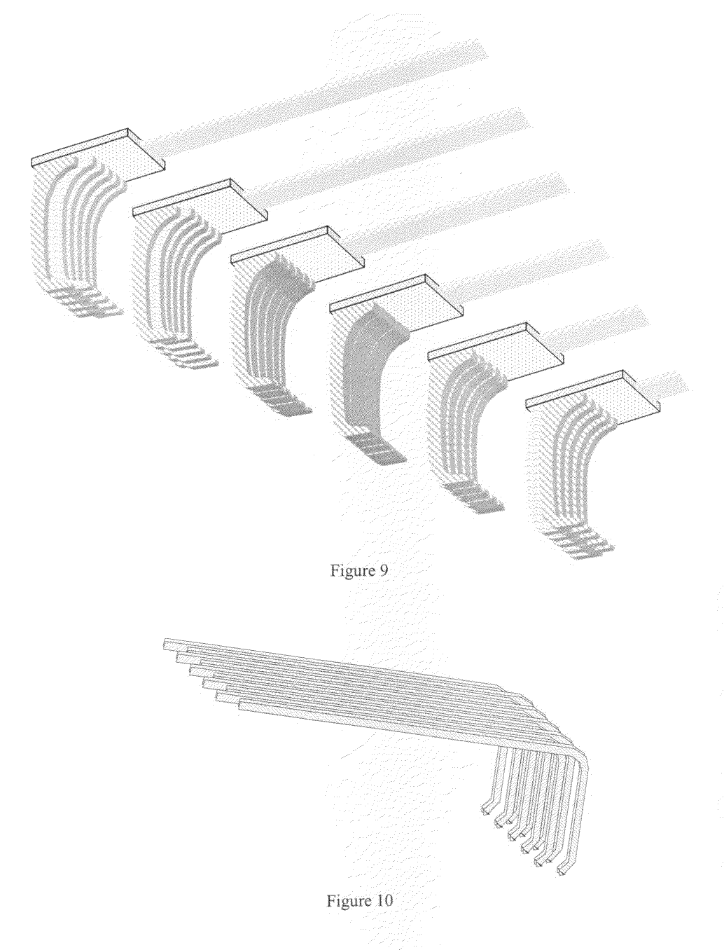

FIG. 8 is a drawing to illustrate a rudimentary version of an assembly using components included in FIGS. 1 through FIG. 6 without showing the peripheral external power- and control-umbilicals necessary for automatic or manual remote control, or appendages such as knobs or levers for changing states of the retractor with manual intervention. Equipped with those, this version of an atraumatic retractor is considered one of the most-preferred embodiments. When transitioning from one state to the other for this two-state retractor, the positions of the retraction fingers effectively become reversed with respect to the retracted tissue, and several stages of this transition are illustrated in FIG. 9. Modifying the design to combine the supporting bars and the retraction fingers could be accomplished with components appearing something like those in FIG. 10.

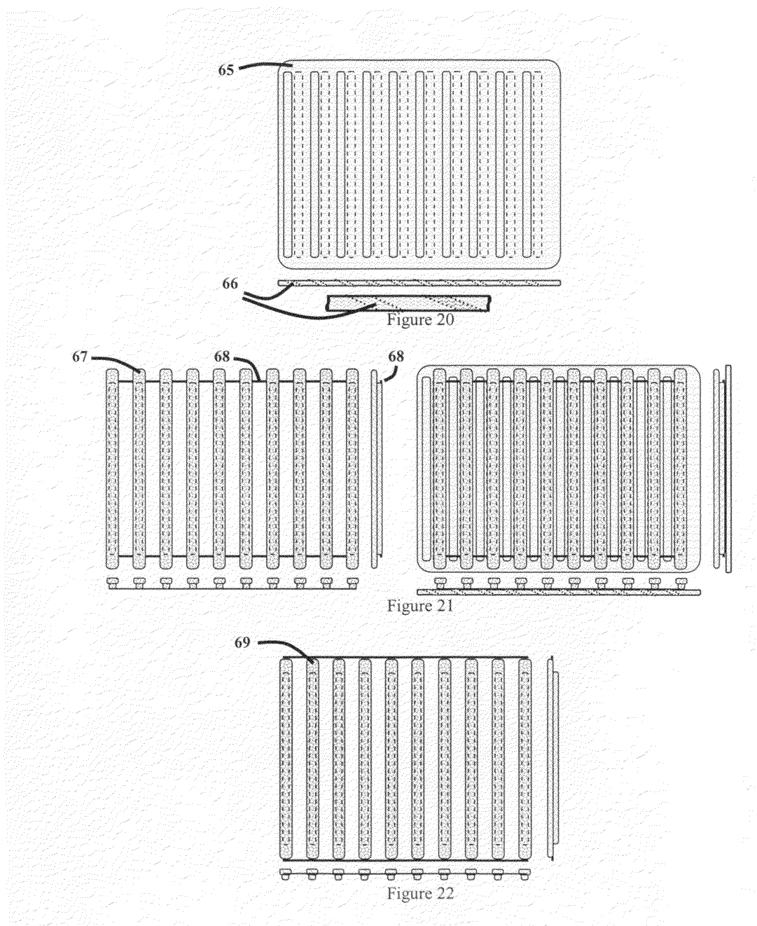

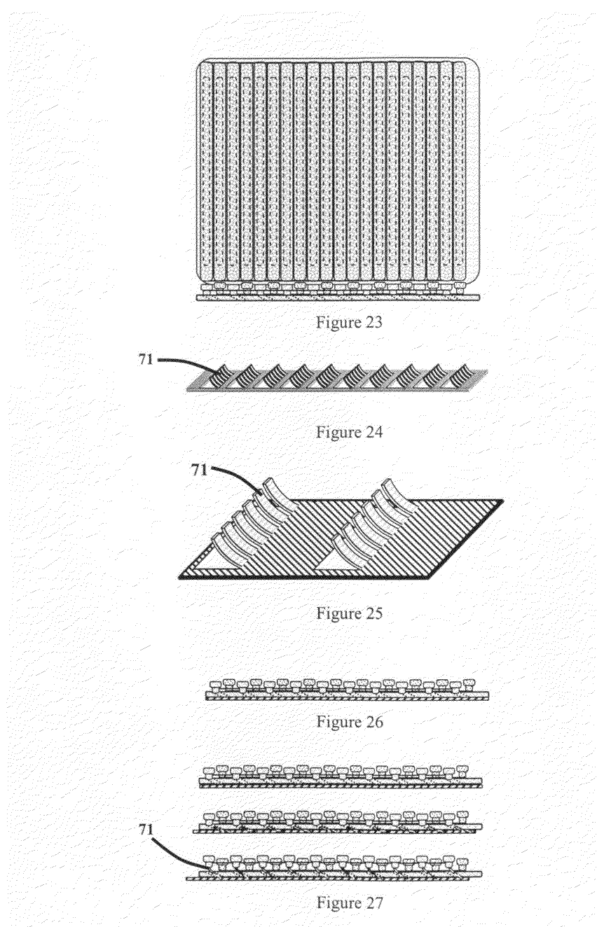

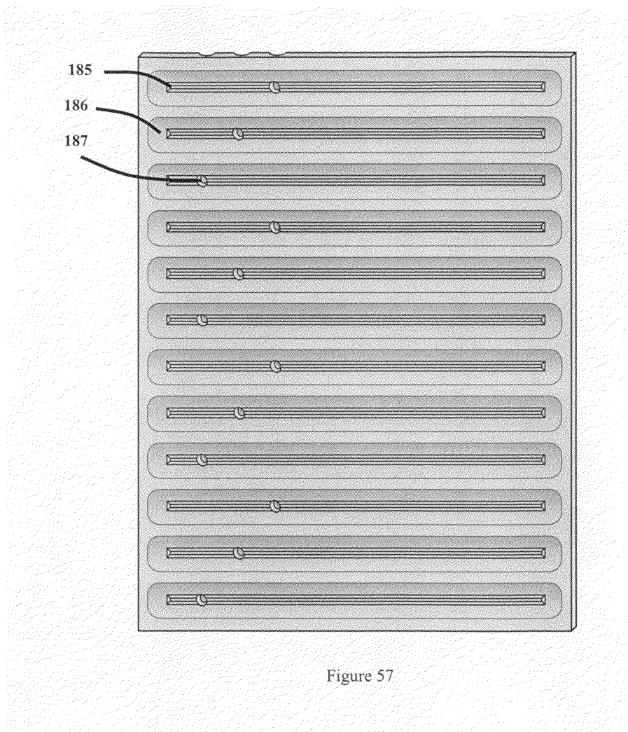

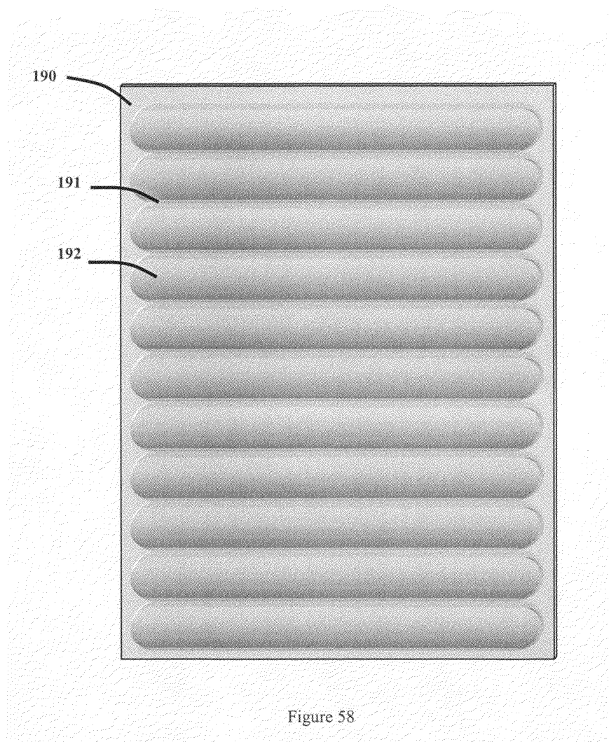

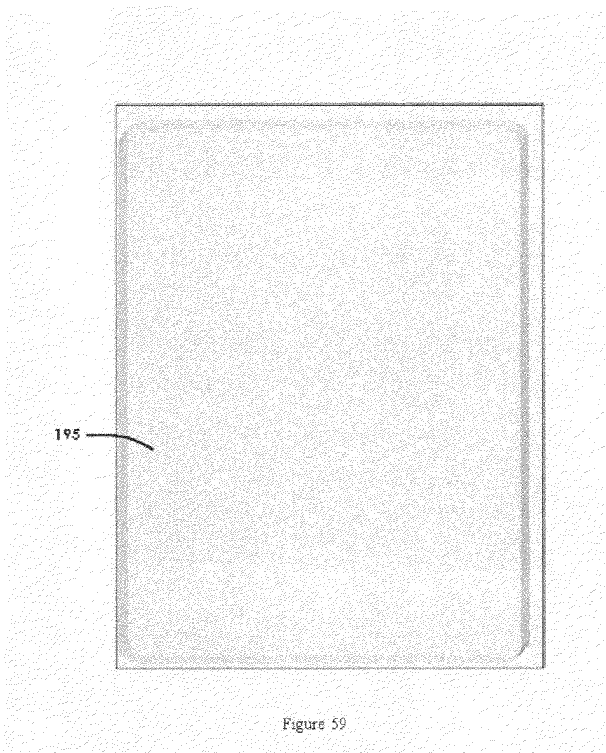

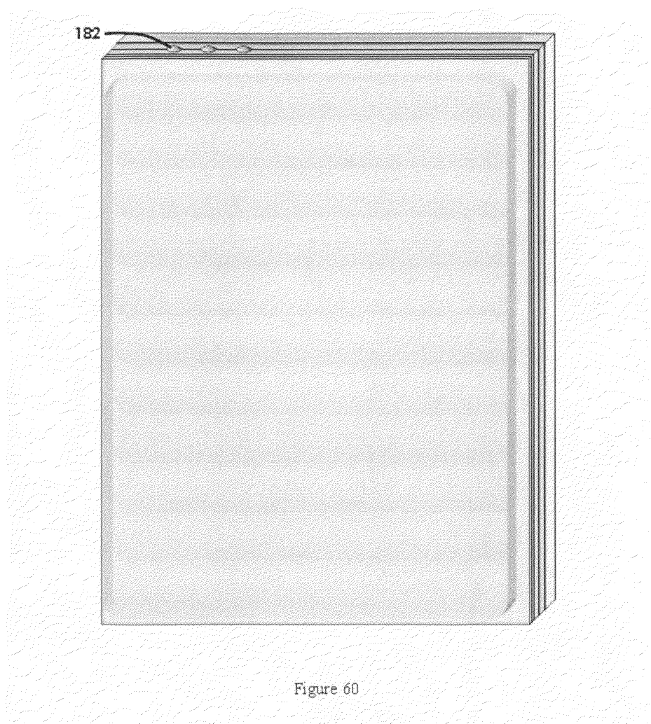

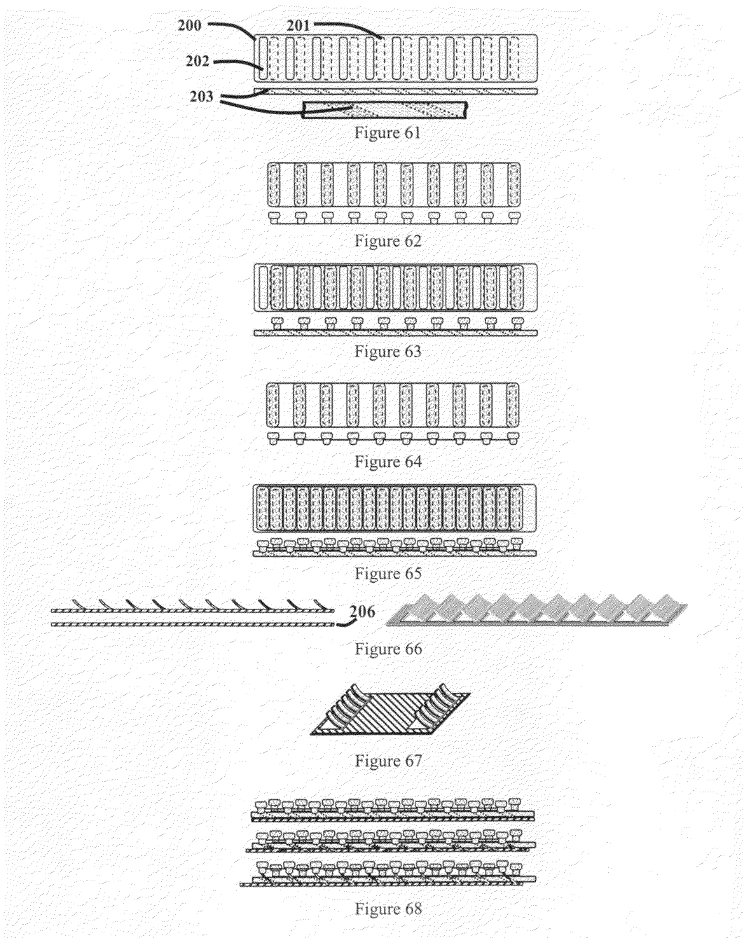

A perhaps equally preferred mechanical embodiment of atraumatic retractor employs a mechanism that can form the bases of not only the “flexible-grate retractor” of FIGS. 20 through 27 and the “flexible-grate brain retractor” of FIGS. 61 through 68, but also the operating mechanism of a body positioning device or a body clamping device as shown in FIG. 78 where its application could obviate the need for “pinning” a patient's skull in brain surgeries. In the first of these embodiments, a fixed-position multi-segment flexible grate 67 having segments held at stable positions by symmetrical guide straps 68 is attached or bonded, along either of the base plate edges adjacent to the slot ends to maintain flexibility, to a flexible base plate 65 with its segments positioned on its upper surface nearly centrally over the locations of long, narrow openings in the base plate's lower surface, as shown in FIG. 21. These openings serve as entry points for diagonal slots 66 in the base plate, the upper openings of all but one of which are at locations midway between the segments of the fixed-position grate 67. A similarly flexible but movable grate 69 has similarly configured segments as shown in FIG. 22. All but one of its segments rest between the segments of the fixed-position grate as shown in FIG. 23, with the lower portions of its segments partly protruding into the base plate's slots, this situation of which, along with its slightly shorter lower regions, positions the upper surfaces of its segments below the surfaces of grate 67, as shown in FIG. 23. Added to this set of joined components is a segmented finger sheet with fingers 71 as shown in FIG. 24, one section of which is shown in close-up view in FIG. 25. This segmented finger-sheet is comprised of a flexible springy material cut and bent to present multiple springy fingers capable of enduring thousands of flattening flexions without breakage. When pressed against the lower surface of the base plate in the position shown in FIG. 26, the fingers are flattened and the relative positions of the flexible-grates' segments remain unchanged. As the segmented finger sheet is pushed or pulled toward the left as shown in the drawing of FIG. 27 by any acceptable means and guided to remain in-line with the base plate by an outer frame or housing (not shown), the individual fingers, separated slightly from each other as they are and thus able to accommodate base plate curvatures, find their ways into the diagonal slots and, upon encountering the lower sections of the movable grate segments, begin to push these segments upward a distance great enough to functionally make their surfaces higher than the segment surfaces of the fixed grate, but small enough to ensure that the segments of the movable grate do not move past the guiding edges of the fixed grate segments. The brain-retractor embodiment shown in FIGS. 61-68 may be understood without further explanation from the preceding description.

Motion of the finger sheet in these embodiments (which may be made without segmented fingers in brain-retractor applications) may be remotely controlled to transition from one state to the other using a sheathed cable (not shown) similar to a speedometer cable with its sheath attached to a protrusion at one end of the base plate and its inner wire attached to the appropriate end of the finger sheet. To “locally” make transitions from one state to the other, a knob or other protuberance such as a lever could be used to activate a mechanism that would cause the finger sheet to move the required amount.

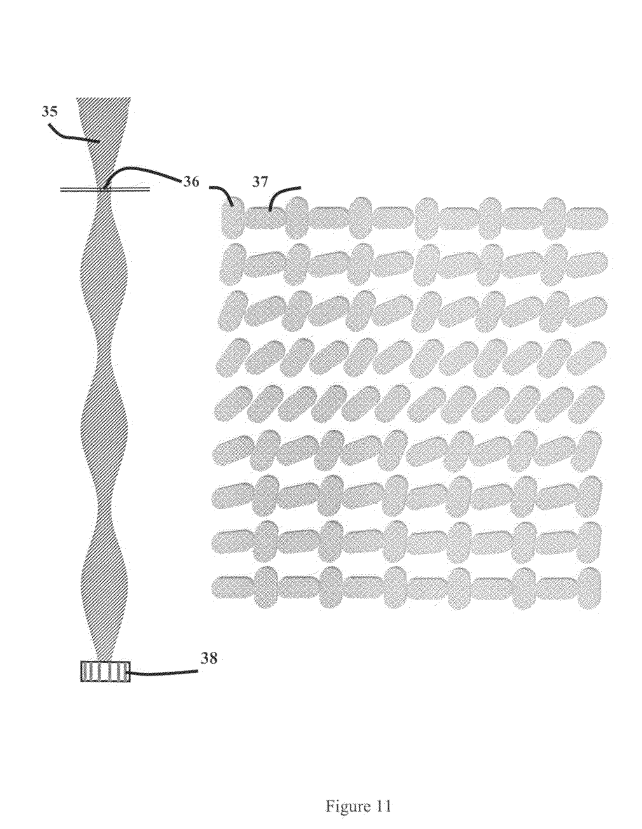

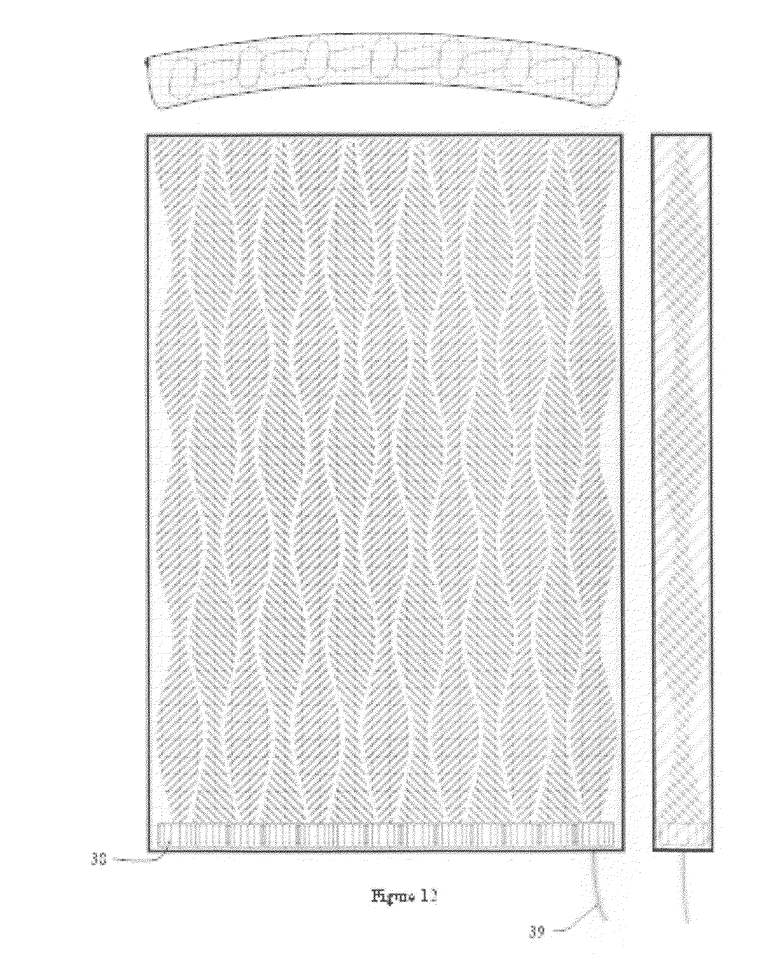

Another mechanical embodiment, perhaps also equally preferred, is a retractor model that presents segment surfaces that move across the retractor face in parallel diagonal directions as its movable elements, comprised of a set of parallel-arranged helix-shaped flexible rods, or more accurately, rods shaped as two-fluted helixes with infinite helical symmetry much like that of a two-fluted drill bit, are rotated. FIG. 11 shows a sectional side-view of this retractor model with its nearest-appearing helical rod 35 shown sectioned axially in the plane of the paper along with a sectional top view of the set of rotating helical components, shown as the top-most row of nine such views, at a fixed position 36 and at the fixed point in time at which the helical rod is shown frozen. The nine sectional top-views show, by time progression of odd-numbered helixes rotating clockwise and even-numbered helixes rotating counterclockwise, how helixes can rotate in positions adjacent to each other without interference if synchronized to have alternately-90-degree-offsets and present surfaces, at positions of equal distance from their ends, that will have distances along lines perpendicular to and closest to their respective axes that describe near-sinusoids, depending on the curvatures of the shank edges, with these same-distance-from-end points on the even-numbered helixes exactly out-of-phase with those of the odd-numbered helixes. With an elastic isolation sheet separating the rotating helixes from retracted tissue, in a way similar to the isolation provided by the outer upholstery material of a back-massaging chair, the peaks and troughs of appropriately-sized rotating helixes will present the maximum-to-near-minimum range of retraction forces and indentation distances that are optimum for tissues ranging from brain tissue to muscle tissue. A helix-element retractor can be operated continuously, acting as an infinite-state, ever-changing retractor, essentially operating as a massaging device, or made to have selected two-, three-, or other multi-state (such as 90-degree) transitions at desired intervals.

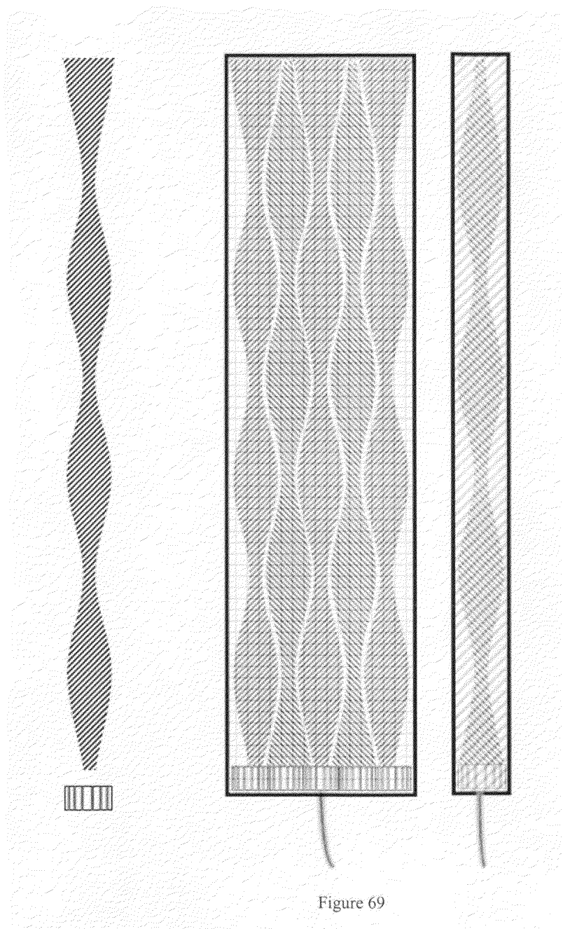

FIG. 69 shows one preferred embodiment of this nested-helical mechanical retractor, this time revealing a much narrower construction to be specifically applied to brain surgeries where damage to brain tissue, some amounts of which are considered to be unavoidable during some procedures, can compromise a person's functional capabilities. As before, gears are attached at the lower portions of the rotating helicals and these may be similarly driven by small sheathed cables to form assemblies that may be made malleable, lightweight, and equipped with mounts that are attachable to conventional goose-neck brain-retractor supports.

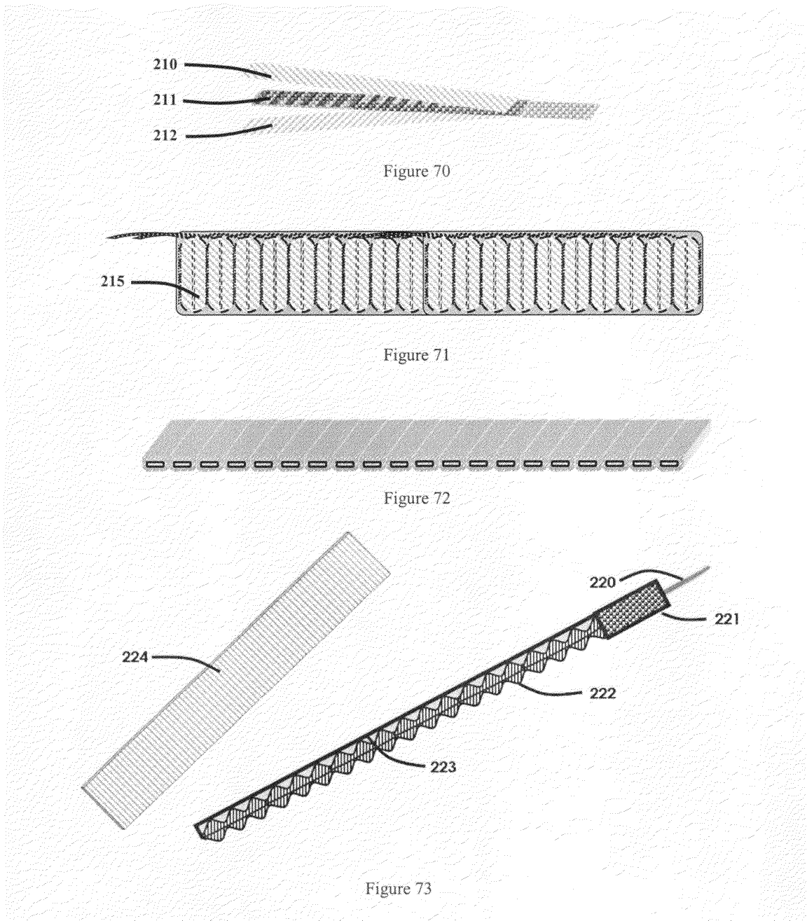

Another mechanical embodiment, perhaps equally preferred for brain retraction, is a retractor model that presents raised segments that effectively move across the retractor face in straight-line directions. FIG. 70 is a drawing that illustrates its basic principle. A thin, semi-rigid strip 211, having affixed to it or fashioned to present a set of preferably evenly spaced zones having raised-relief profiles, is guided to slide between an elastic isolation membrane 210 and a semi-rigid strip 212, both shown separated from strip 211 at one end to distinguish them as separate components. The profile of strip 211 resembles a well-known rack and for this reason this type of retractor is termed a sliding-rack retractor. Strip 212 may comprise the flexible and frequently malleable component of a conventional brain retractor, or it may be a separate isolation strip to make the assembly a more-easily fabricated consumable item. The raised-relief sections preferably have the profile of speed bumps spaced on strip 211 to appear, in a side view, to have an outline resembling the positive values of a sine wave curve. In use, semi-rigid strip 212 is placed in a fixed position such that the upper-shown surface of membrane 210 contacts and applies retraction pressure to the tissue to be retracted. Strip 211 is then moved along a pathway in a reciprocating fashion, preferably guided by the inner sides of the retractor's construction, at appropriate speeds and dwell-times and in directions parallel to the edges of strip 212 and membrane 210, making a peak-to-peak excursion of at least half the distance between the centers of the raised-relief zones. Lubrication of the inner surfaces with material approved for the application is preferably added to reduce friction and enable uncompromised movement, and the material of strip 210 is selected to have sufficient rigidity to both resist the pulling and stretching that could prevent proper operation under any useful retraction pressure at any point during its usable lifetime, and prevent excessive lateral movement of the retracted tissue when the retractor is transitioning between dwell or maximum excursion states.

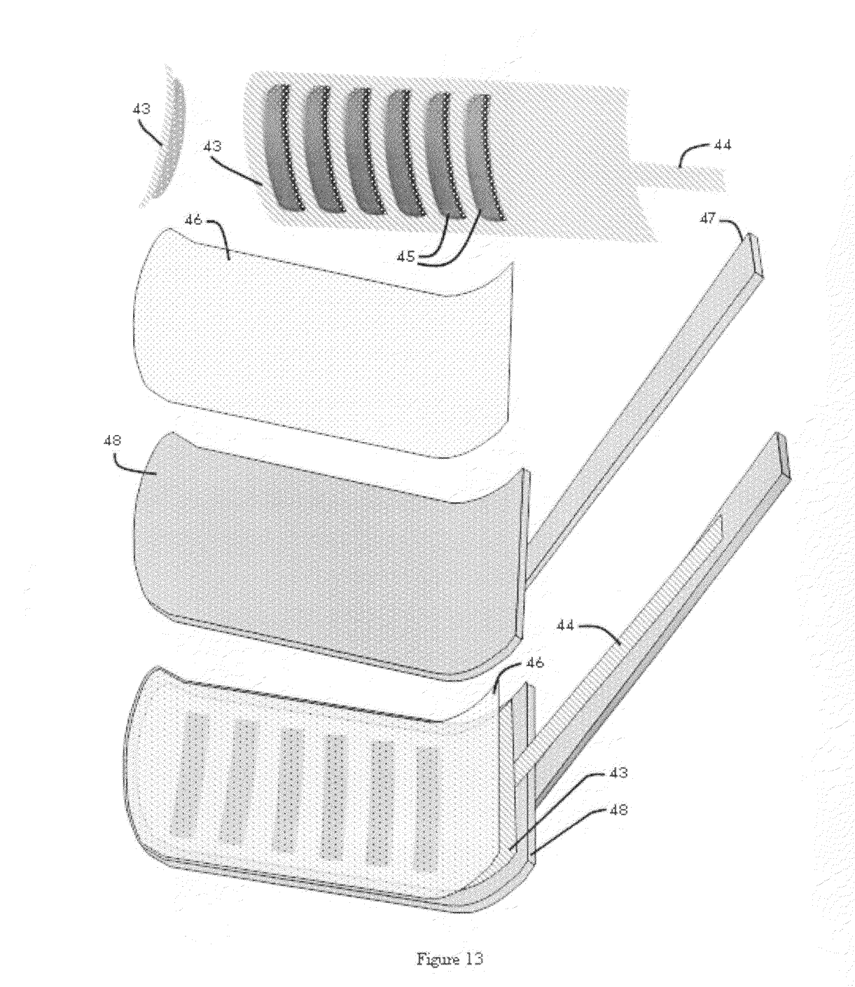

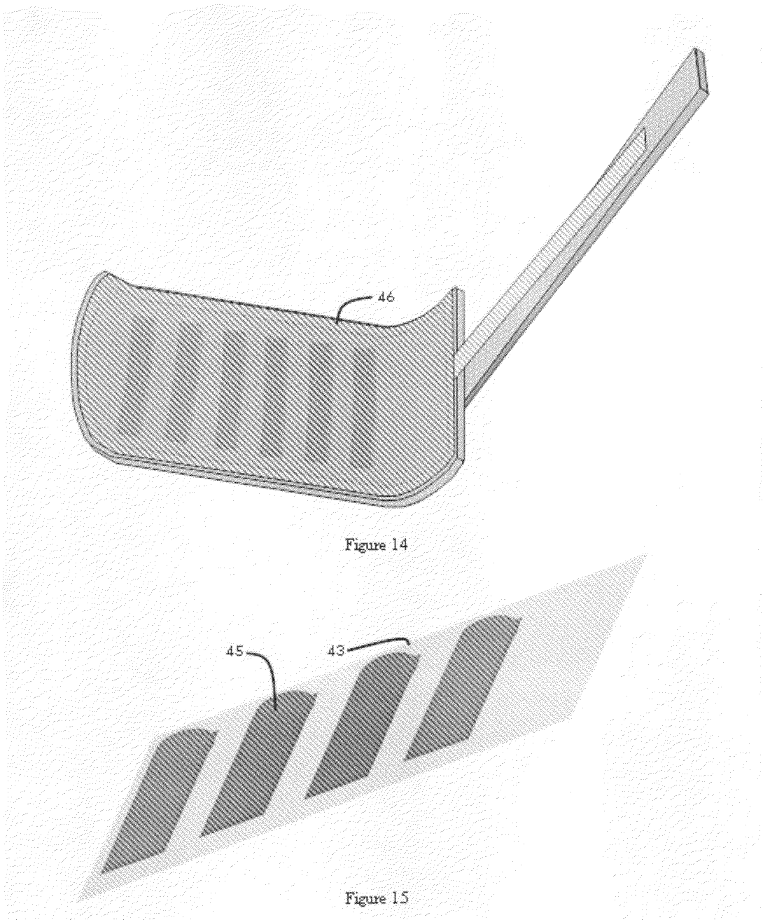

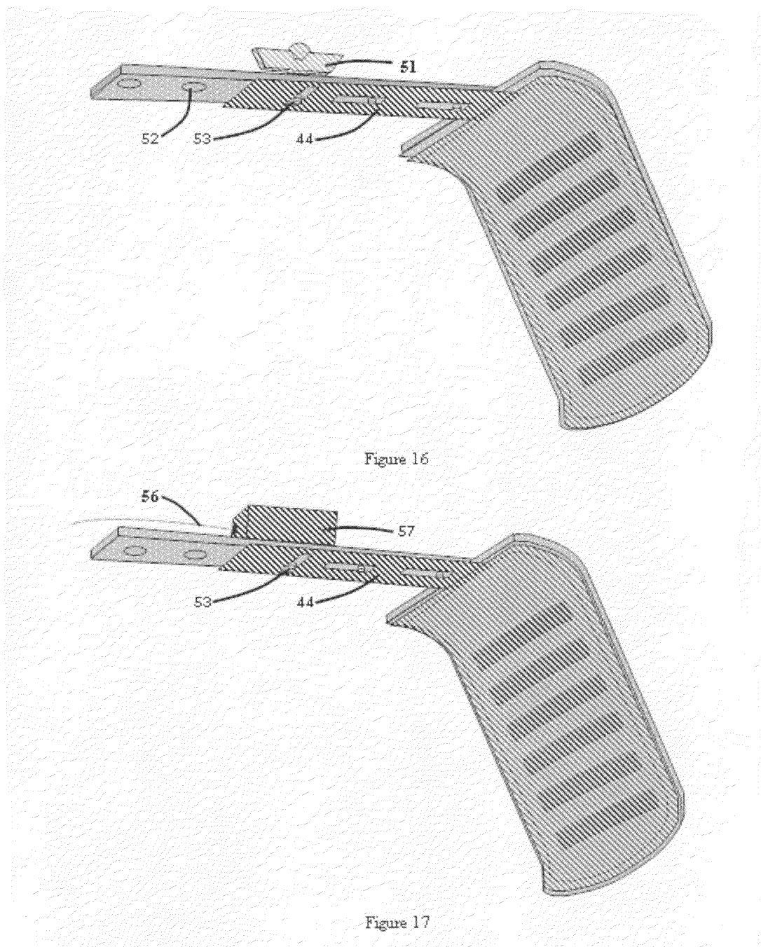

Somewhat more demanding applications for a sliding-rack retractor are regions where retraction pressures are higher than those used for brain retraction. FIG. 13 is a drawing of similarly functioning components of a larger such retractor where the analog of the conventional brain retractor “blade” is shown here as a conventional retractor blade 48, the analog of the elastic membrane is the covering sheet 46, and the sliding semi-rigid strip is a wider, semi-rigid flexible strip 43 having raised sections 45 and a flexible protrusion 44 for reciprocatingly driving it. Supporting bar 47 allows the assembly to be attached to a support structure for stability. An example of an assembled unit, with covering sheet 46 attached to the edges of the supporting blade 48 is shown in FIG. 14. A drawing to illustrate the profile of the raised sections is shown in FIG. 15. For manual operation, this assembly can incorporate a knob 51 that can drive a cam 53 that rides in a slot in slider extension 44 as shown in FIG. 16. Hole 52 is one of two that allow the retractor to be directly or indirectly secured to a support structure.

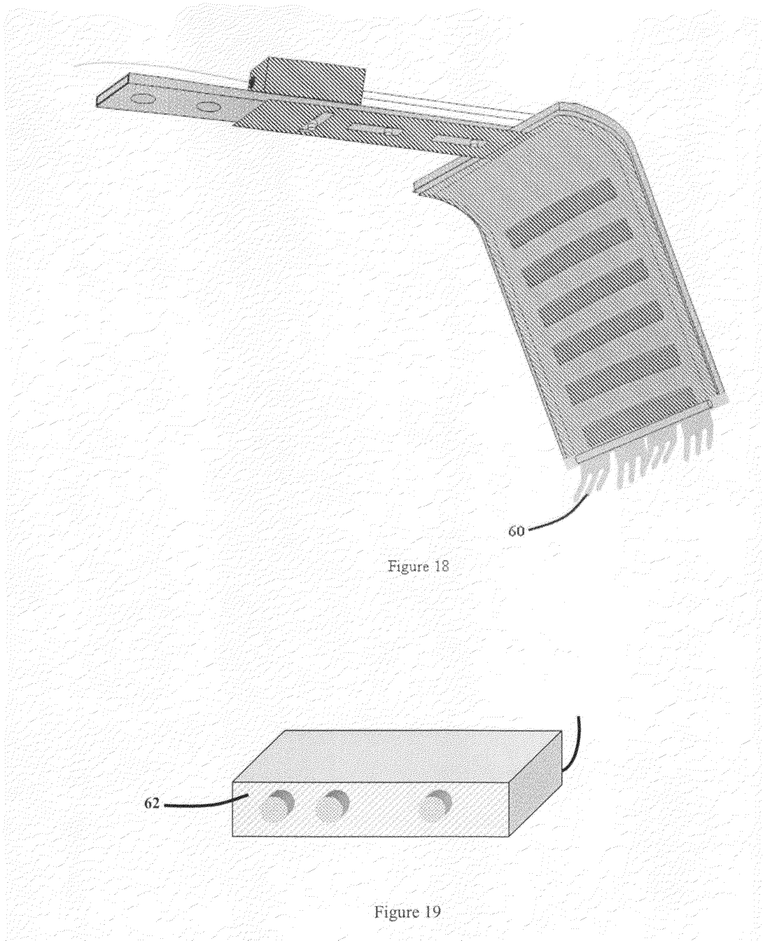

The drawing of FIG. 17 shows an example of a modification that can be made to the sliding-rack retractor of FIG. 16 to enable actuation by remote control. A mechanism within assembly casing 57 drives the cam with power supplied through an umbilical 56 consisting of, for example, electrical wires powering a motor or solenoid, a sheathed cable like a speedometer cable having an inner wire that rotates or moves in translational directions, or flexible tubing that conducts fluid to power a cylinder or fluid motor, or alternately, directly drives the slider extension 44. For retractors having teeth along their lower surfaces, anticipating the desirability of applying and removing the forces they might add to retracted tissues prompts visualization of a means for withdrawing or reciprocally applying them to the tissue, and this possibility is addressed in FIG. 18.

Operation of any of the aforementioned mechanical retractors or the head-clamping device requires a power source and a control means, of course, and although mentioned elsewhere, with a range of potential means listed, FIG. 19 acknowledges this need by representing a unit, preferably to be located out of the sterile field, that can serve these functions. Also mentioned elsewhere are the major anticipated outputs and control means; namely, electrical power, mechanical motion, or fluid motion or pressure alteration, with control supplied by timer, microprocessor, computer, or the like.

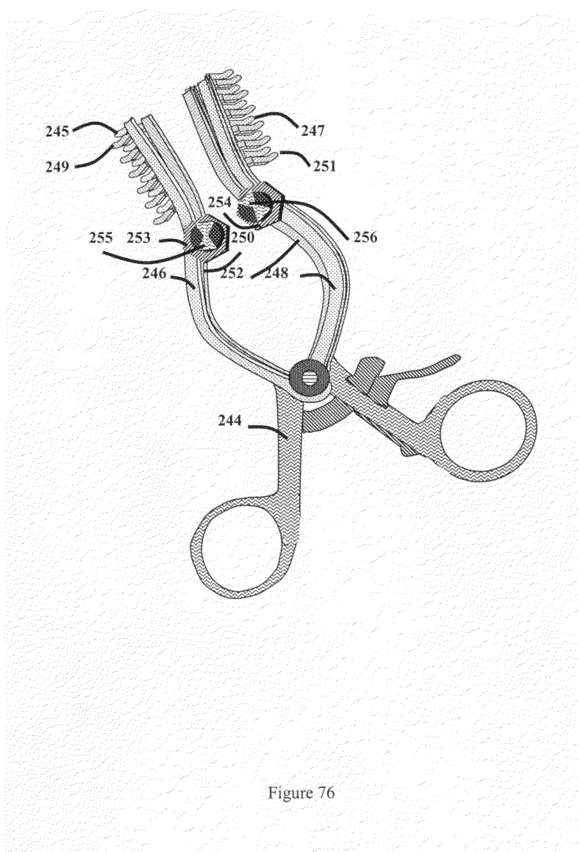

The atraumatic retraction technology within the scope of this invention can also be applied to other retractor designs, including existing devices, one example of which is the well-known Weitlaner self-retaining retractor, the basic construction of which is shown in FIG. 76 by the handles and locking mechanism 244 generally depicted by all component parts below the common pivot point that is central to the arms and handles in FIG. 76, the set of retracting teeth 245 along with its support arm 246 shown on the left side of this rendering, and the set of retracting teeth 247 along with its support arm 248 shown on the right side of this rendering. To enable the standard Weitlaner self-retaining retractor to become an atraumatic retractor, added are a third set of retracting teeth 249 along with its support arm 250 positioned below support arm 246, a fourth set of retracting teeth 251 along with its support arm 252 positioned below support arm 248, locking mechanisms featuring, as a preferred example, cam-support 253 that is attached to support arm 246, cam-support 254 that is attached to support arm 248, and their respective and associated cam-rotating knobs 255 and 256 that clamp arms 250 and 252 in positions in which their supported tooth-sets 249 and 251 are thrust outward to assume positions farther-apart than the formerly-more-widely-separated tooth-sets 245 and 247 when rotational forces are applied to knobs 255 and 256 causing their attached cams to rotate against the facing surfaces of lower support arms 250 and 252 until said cams come to rest within detents in these surfaces, said detents which may be formed with unequally angled slopes to allow easy entry and withdrawal of their associated cams from one direction and prevented withdrawal from the other.

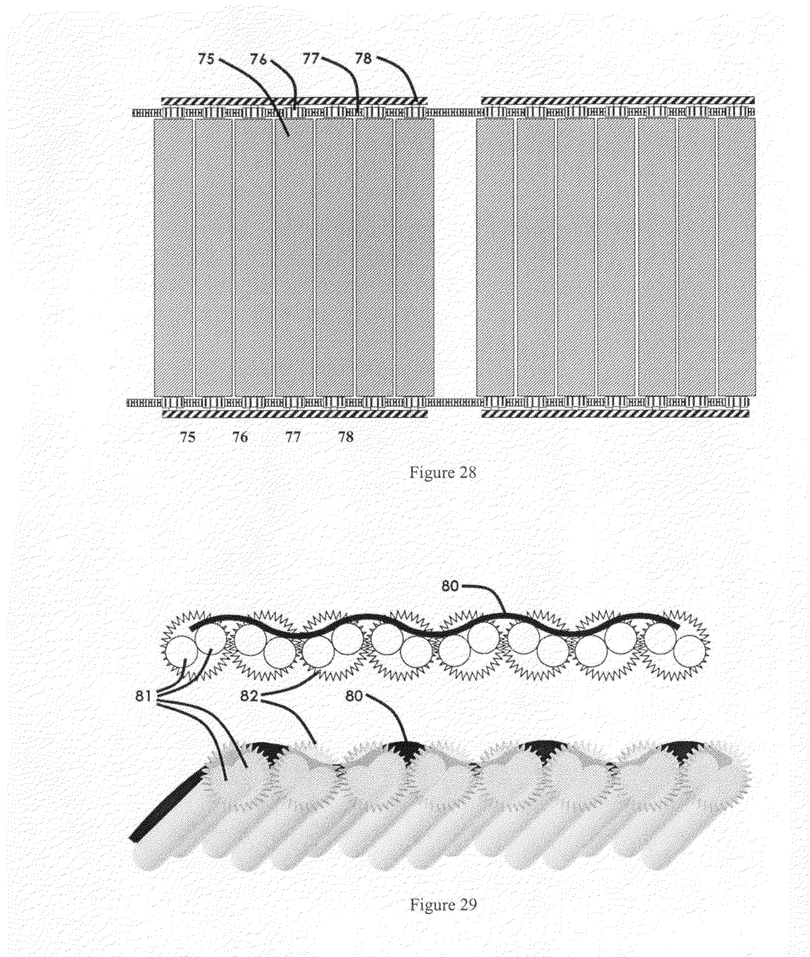

To illustrate yet another mechanical option for shifting pressure among regions of retracted or supported tissue, the drawing of FIG. 28 shows two sets of rollers, one roller 75 of one set of which can be seen to have a gear 76 mounted at its upper end and a similar gear mounted at its lower end, both preferably to a solid axel that terminates at each end into a side of a frame 78 (only the sides of which are shown) that maintains the relative positions of the rollers and provides an attachment point for applying lateral forces to one roller-set (in an arrangement where transition power is applied differentially between this frame and the corresponding frame supporting the second roller set), with the upper gear riding against a rack 77 and the lower gear similarly riding against a rack, both racks of which comprise a support structure against which force may be applied to enable the nearest-appearing sections of the rollers to apply pressure to tissues against which they may be held. With the other set of rollers similarly disposed into a frame and against the mentioned rack, one can visualize the sets of rollers moving toward and away from each other to cause the regions of applied pressure to shift laterally while the roller positions transition between one state and a second state, state positions of which would preferably correspond to positions separated by a distance equal to the separation distance of two rollers within a single set. To allow for more curvature along the vertical dimension than these straight rollers would allow, the rollers may be shortened to any length and multiple such sets having these new lengths could be stacked to have axel axes that would be parallel to the associated tissue section of each roller set. To allow for curvature beyond what a straight rack and straight frame would allow, the rack and both frames could be made curved or flexible to accommodate the curvature of the tissues involved. Again, an isolation membrane would likely be desirable in this situation.

A modification of the roller-based atraumatic retractor uses arrangements of rollers in triad configurations, each having a common axis around which each can rotate to present roller surfaces that always transition in one direction for the purpose of preferentially stimulating blood perfusion in the same direction in which the rollers transition.

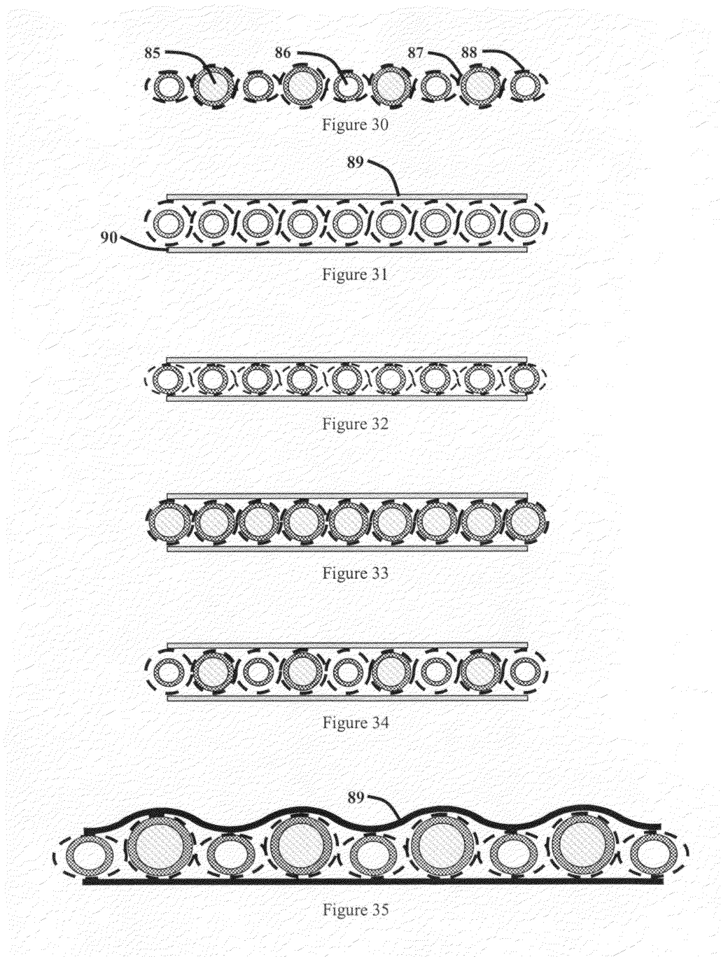

Still other mechanical configurations achieve such shifts, one final example of which is shown in FIG. 29 where pairs of posts 81 attached to interconnecting gears 82 are caused to rotate about midpoint axes in alternating directions. An isolating membrane 80 helps to smooth pressure-applying surfaces as the orientations of the posts transition reciprocally between, as an example, 45-degrees counterclockwise from, to 45-degrees clockwise from a position in which the presented co-tangential surfaces of the posts describe a flat plane.

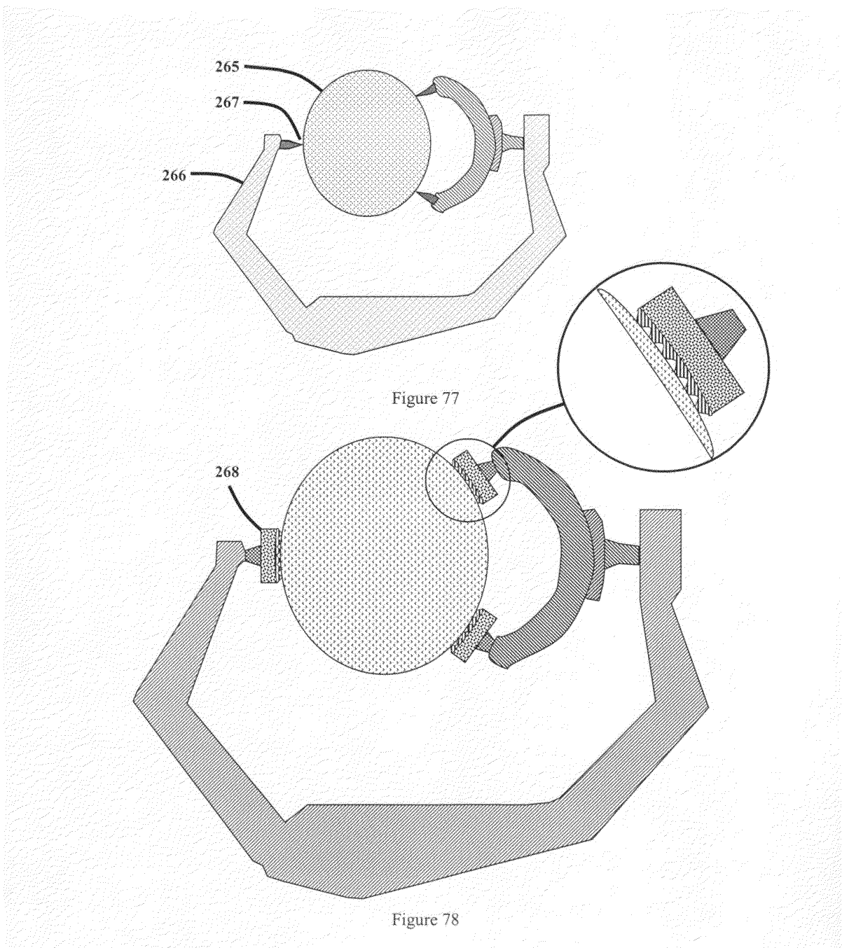

Perhaps the most important application of the atraumatic technology is the tissue-positioning device. Taking the place of the skull-piercing pins 267 of the Mayfield Skull Clamp 266 shown protruding into the skull of a patient's head 265 in FIG. 77 are three-each of the atraumatic head-clamping device 268 shown most clearly in the magnified view at the right-side of FIG. 78 comprised of either a mechanically operated model of the present invention such as the atraumatic retractor mechanism on which the flexible-grate retractor is based, or a fluid-operated model of the present invention such as the atraumatic retractor assembly on which the limited-expansion-chamber retractor shown in FIG. 54 is based. For this application, any model and design featuring this technology will be sufficient to cyclically relieve pressure at the tissue-positioned, or clamped regions, while strictly maintaining the position of a patient's head so as to not compromise its alignment with the display or other aspect of physiology-mapping instrumentation, over the course of many hours, provided that cycling of the pressure-applying retractor segments is accomplished in such a way that pressures are not relieved at any of the tissue sections over the course of its cyclical operating period until pressures applied to all complementary sections are fully restored. Detailed operation of the limited-expansion-chamber retractor is discussed below.

Designs of fluid-operated atraumatic retractors rely on components that partly or entirely undergo a change (size, shape, position) through the influence of a change in a fluid (pressure, volume). The simplest design incorporates tubing that can be made to expand. In a cross-section view, FIG. 30 illustrates changes in tubing diameters, and therefore outer-wall positions of alternate sections of tubing disposed in an array that could be placed between a solid surface and a section of living tissue. Such an array can be formed from two lengths of identical expandable tubing laid “back and forth” onto an existing retractor blade, for example, and cross-section view of this array might assume the appearance of the drawing after one length of tubing was subjected to higher fluid-pressure. A problem arises, however, when differences in loading, or opposition forces at the outer walls of these tubes cause ballooning of less-loaded or unloaded sections since this can both limit the pressure increases that are desired at adjacent tissue surfaces and create a risk of rupture in ballooned areas. To prevent such occurrences, a flexible constraining component 88 comprised of material such as fabric can be placed around each tube to prevent excessive expansion. The constraining components may be interconnected to remain loosely in position around non-expanded tubing sections carrying, for example, low pressure fluid 86, whereas expanded tubing sections carrying fluid 85 at pressures sufficient to expand their outer walls to diameters larger than their constraining components will permit will be bound by the constraining components shown to be unyielding as in position 87. FIG. 31 shows a similar cross-section view where all tubing sections are unpressurized. An elastic isolating membrane is represented by a flat sheet 89 and a solid surface, such as a flat retractor blade, is represented by a flat plate 90. Once an apparatus like this example is placed in position against tissue that is to be retracted, and light retraction pressure is applied, the cross-section view of FIG. 32 illustrates the status of each tubing section and its associated constraining component. FIG. 33 illustrates the change in this view's appearance when all tubing sections are subjected to pressures sufficient to expand them to the diameters of the constraining components. FIG. 34 depicts a similar view when no retraction pressure is applied and one of the tubing lengths is unpressurized, and FIG. 35 illustrates representative conditions of the tubing sections when this example two-state retractor is in tissue-retracting position, in one of its two states and an isolating membrane 89 is disposed between the tubing-section retractor-segments and the retracted tissue (not shown, but everywhere contacting the upper-shown surface of the isolating membrane 89). For all fluid-operated atraumatic retractors, the fluid may be gas, where priming of the tubing and chambers is obviously unnecessary. When liquid is used, however, except for perforated-chamber designs discussed later, expelling or withdrawing with partial-vacuum most or all of the air or other gas which may remain before introducing liquid into these components is a preferred method of operation. Using liquid for chamber expansion is considered preferable in some applications since expansion with controlled volumes of liquid is generally less of a problem that gas could be in a burst situation.

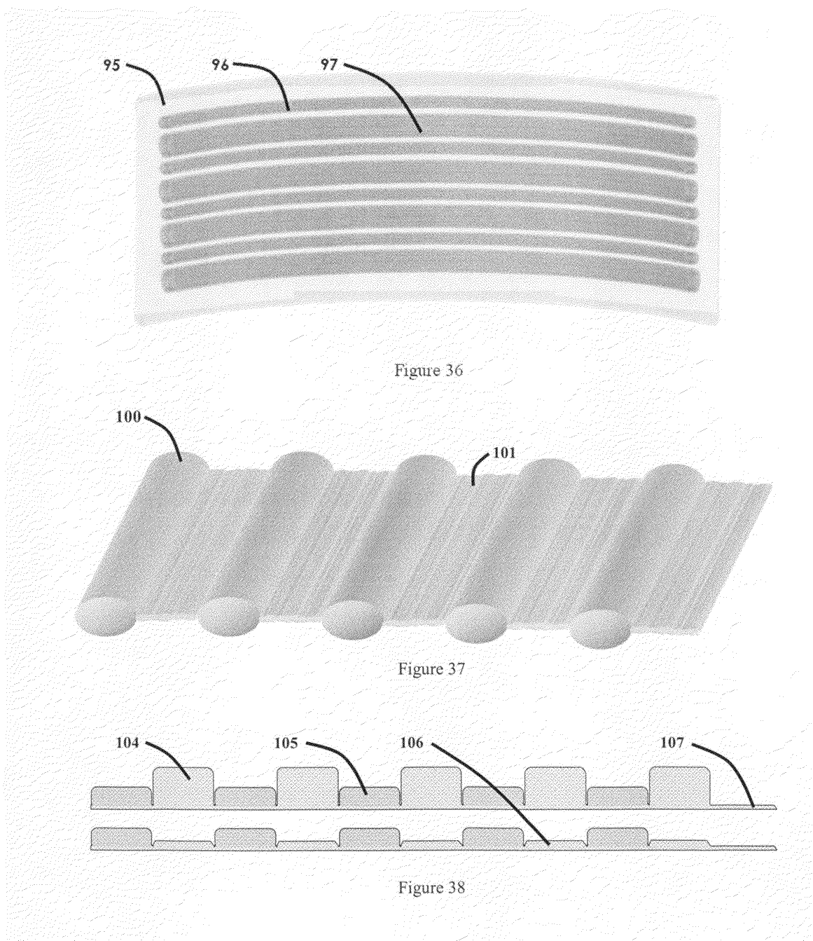

Constraining components become unnecessary if the expandable tubing depicted in FIG. 36, for example, is replaced by inelastic tubing composed of materials that are substantially not expandable, an example of which is the well-known wire-splice-covering-and-insulating products known by the term “shrink sleeving”. FIG. 36 illustrates an arrangement of lengths of such inelastic tubing, half of which are shown in a state 97 as they would appear if either pressurized or subjected to ambient pressure, and half of which are deflated (e.g., at 96) by the application of a partial vacuum, disposed against the convex surface of a wide retractor blade 95. Again, two sufficiently long lengths of such tubing could be used to accomplish the intended atraumatically retracting function, or lengths like those shown interconnected and ported as necessary with manifolds and end seals.

FIG. 37 illustrates alternately inflated and deflated sections, 100 and 101 respectively, of inelastic chambers that could be fabricated as an extrusion, thereby simplifying construction of tissue-supporting and retracting devices to benefit mass-production.

FIG. 38 illustrates a construction of chambers comprised of inelastic material that are interposed between substantially solid segments 105 whereby pressure zones may be alternated, achieving essentially the same purpose as those of earlier two-state-retractor examples, by deflating the chambers to have segment-surfaces protrude a shorter distance 106 from the plane of its immovable support to achieve one state, and then inflating the chambers to force the segment-surfaces to protrude past the substantially solid segments to have positions at a greater distance 104 from this plane. Inner channels can interconnect the chambers and be fed by a tube or other hollow protuberance 107.

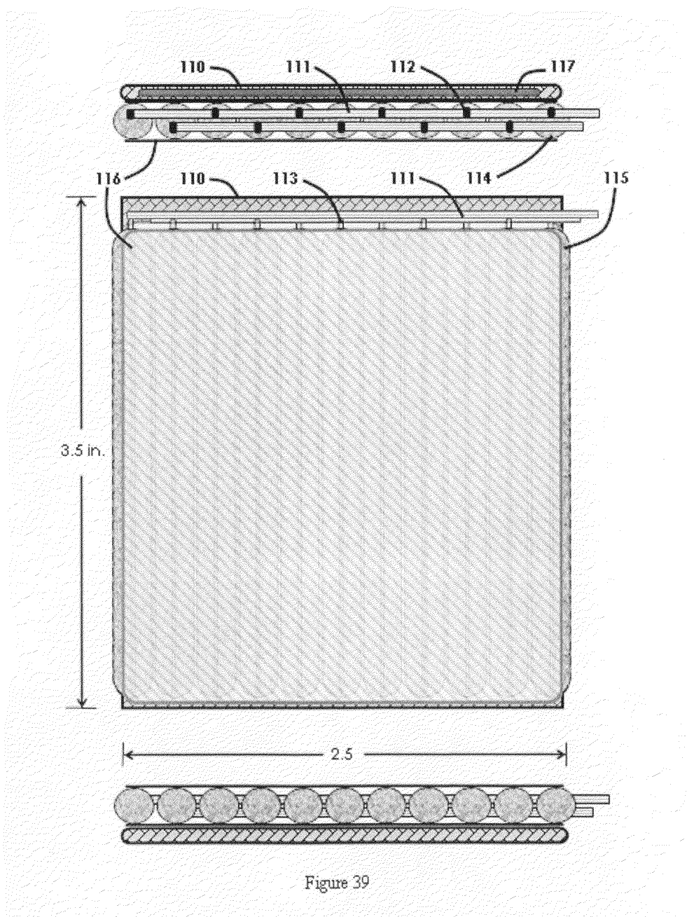

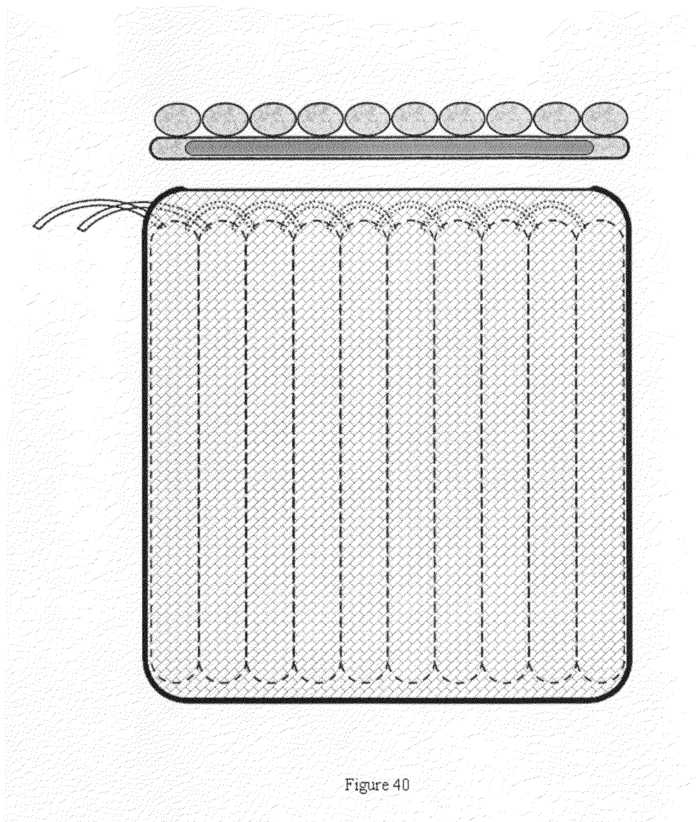

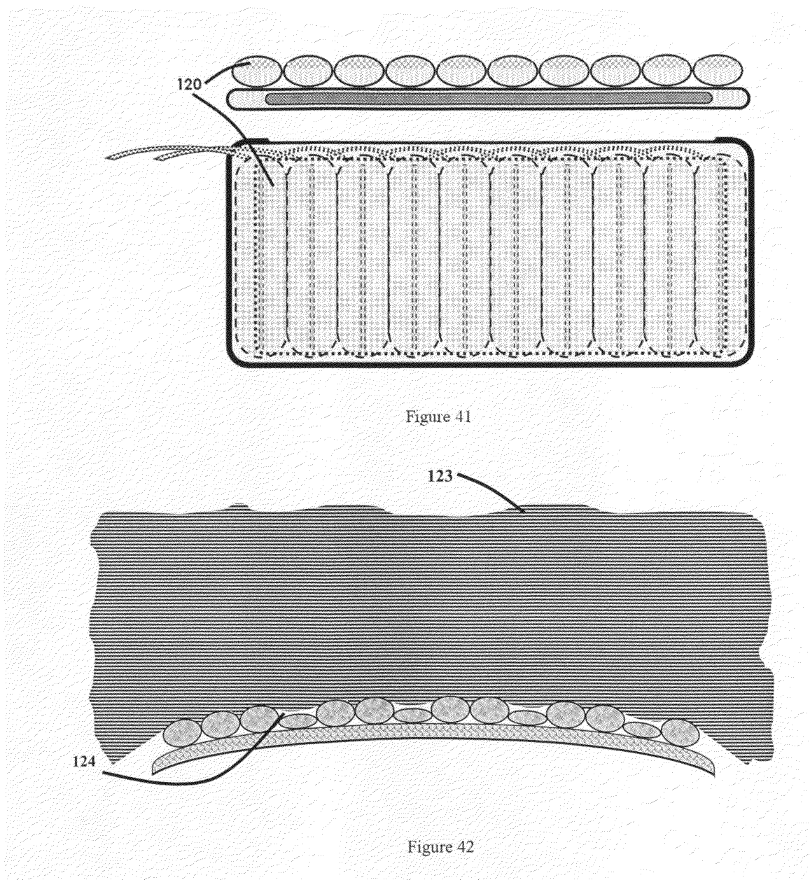

FIG. 39 illustrates a usable configuration for a fluid-operated retractor that is designed to incorporate a kind of glove 110 having a cavity with an opening 117 of length slightly shorter than the width of a preferably existing retractor blade with which its use is intended, and which is formed from material that can elastically fit-over, conform to, and be held by, in this example, an existing Kelley retractor blade 110. The chambers of this retractor are comprised of flexible inelastic tubes or preformed chambers 115 that are connected at one end 114 to other tubes and/or manifolds 111 by interconnections 112 and 113 within their groupings and through preferably small substantially inelastic tubing to at least one source of fluid and any necessary valves, pumps, and controlling means by which the chambers can be subjected to changes in volume and/or pressure thereby exerting retracting pressure through isolating membrane 116 to a region of the tissue to be retracted. FIG. 40 is a rear view of a similar configuration using components that are differently interconnected and considered more amenable to molded-fabrication. FIG. 41 represents a similar design, with interconnecting components not shown, configured to fit a wider and more shallow blade. In this embodiment, however, the forward-surface sections of the retractor segments have perforations that enable the pressurizing fluid, which could, for example, be oxygen or oxygenated blood, to escape from the chambers for the purpose of aiding preservation of the tissue by bathing the surface of the retracted tissue, or, with the addition of a thin permeable or perforated structure (not shown) bearing protrusions that can break the surface of the tissue, by both bathing the surface of the retracted tissue and enabling injection of the pressurizing fluid into subsurface regions of the retracted tissue.

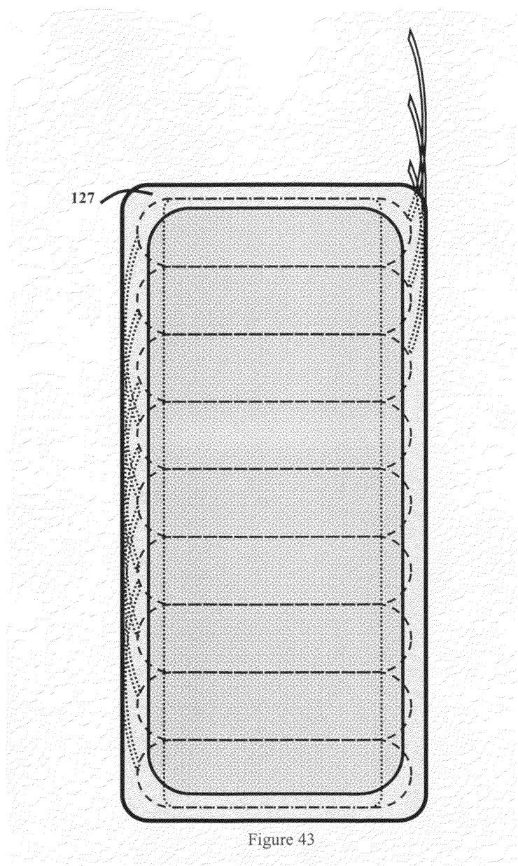

FIG. 42 illustrates an exemplary profile of tissue 123 that is under retraction by a three-state atraumatic retractor that creates zones of reduced pressure 124.

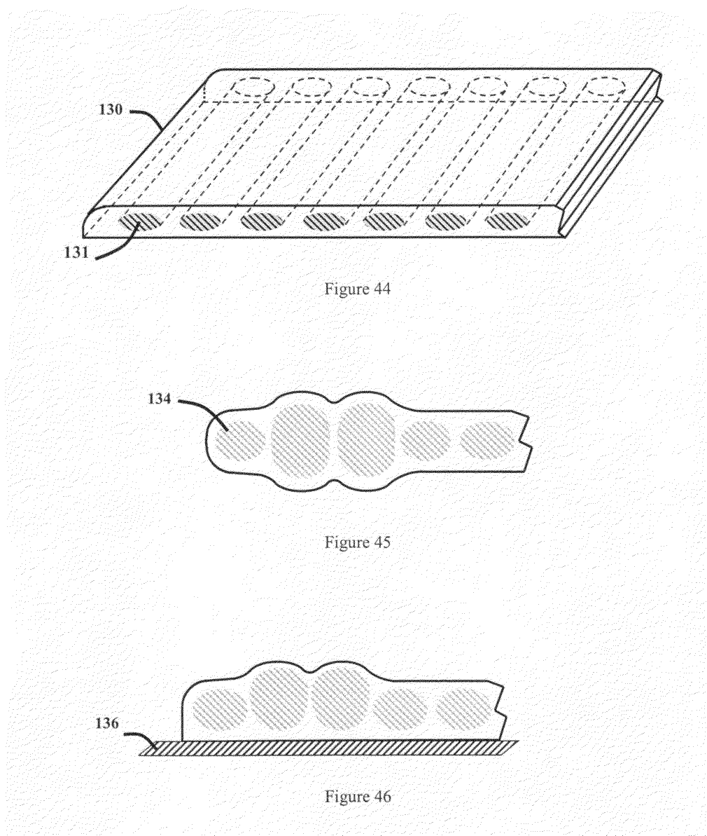

FIG. 43 illustrates a usable configuration whereby the retractor can remain attached to a retractor blade by an elastic section 127 of a covering intended to be stretched over the blade in much the same way that a fitted sheet covers a mattress.

FIG. 44 illustrates an extruded component 130 which may be cut to lengths and widths to fit various existing retractors or other surfaces to lower production costs. As with other extrusions the material is preferably an inelastic flexible material which, in this configuration, will allow areas of depression or deformation when chambers 131 are unpressurized, and areas of potential shape-change when these chambers are pressurized, as FIG. 45 illustrates with a similarly-formed extrusion showing hollow sections 134 at one end of the extrusion and with pairs of inflated and deflated chambers that show a way that effective chamber width may be adjustable to meet different applications, and as FIG. 46 illustrates showing an approximate cross-section appearance when the atraumatic retractor section is under load between tissue and a supporting back plate.

FIG. 72 illustrates a similar but much smaller extrusion that can be used for brain retraction. With appropriate manifold-attachment to one or both long edges, as convenient, multiple parallel expandable chambers may be presented in a lightweight, thin construction. In the form shown in the drawing, the extrusion is meant to be attached to a conventional brain retractor and held with a double-sided adhesive material.

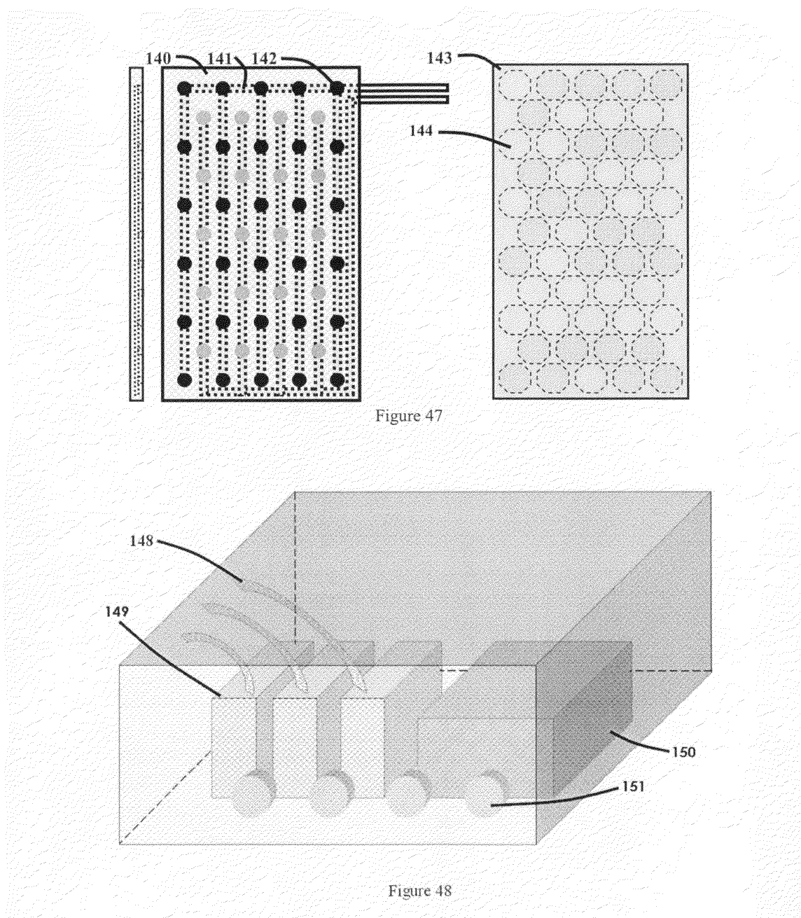

FIG. 47 illustrates components of another two-state atraumatic retractor having a compound-layer assembly 140 of molded sections wherein channels 141 conduct the working fluid to ports 142 that present fluid that are covered by a bubble-bearing flexible inelastic covering 143 is bonded ultrasonically or by other means to assembly 140 at every contact point, or essentially at all areas not within the bubble sections 144.

In all of these fluid-operated assemblies, as with the mechanical devices described earlier, a power-sourcing and controlling apparatus is required to drive the devices to transition from one state to another and maintain conditions necessary to sustain these states. FIG. 48 illustrates an exemplary device to perform such functions, in this case containing output ports 148, control valves 149, and a pump 150. Controls to adjust, actuate, select, and turn-off these functions are represented by knobs 151 with which a human operator may interface.

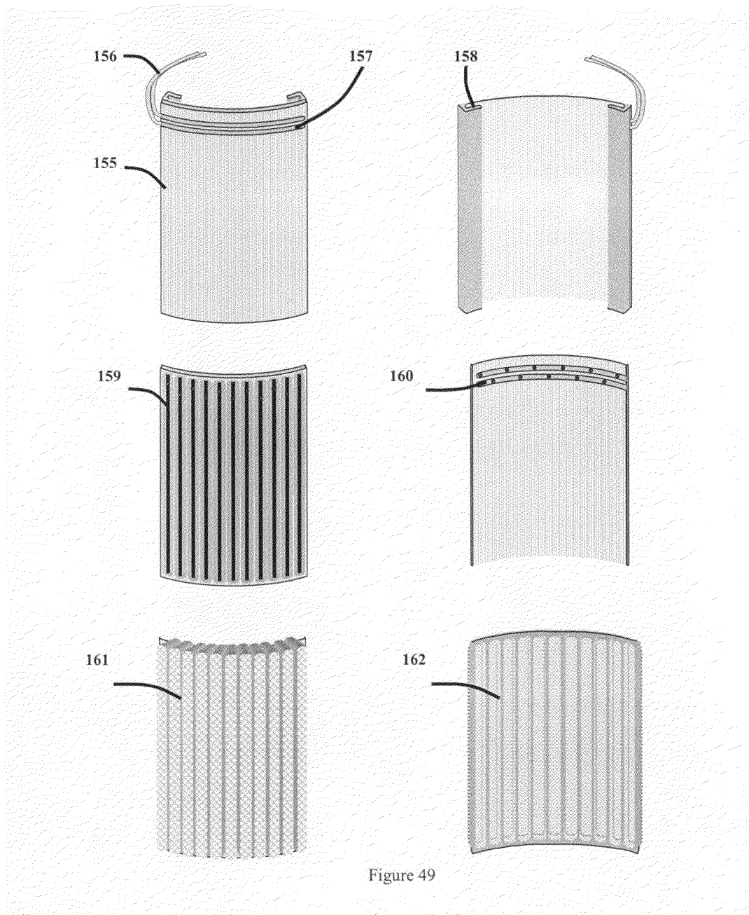

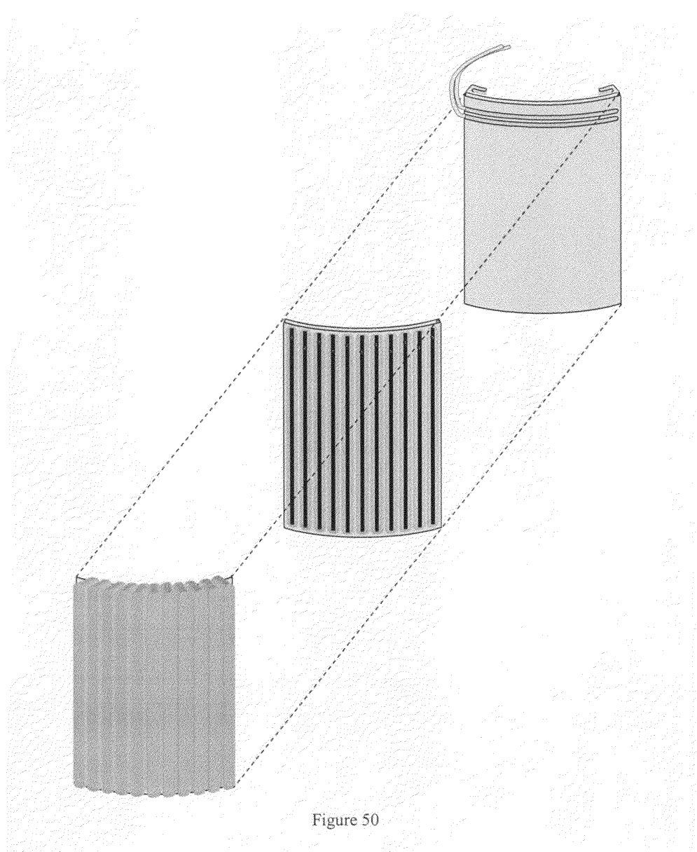

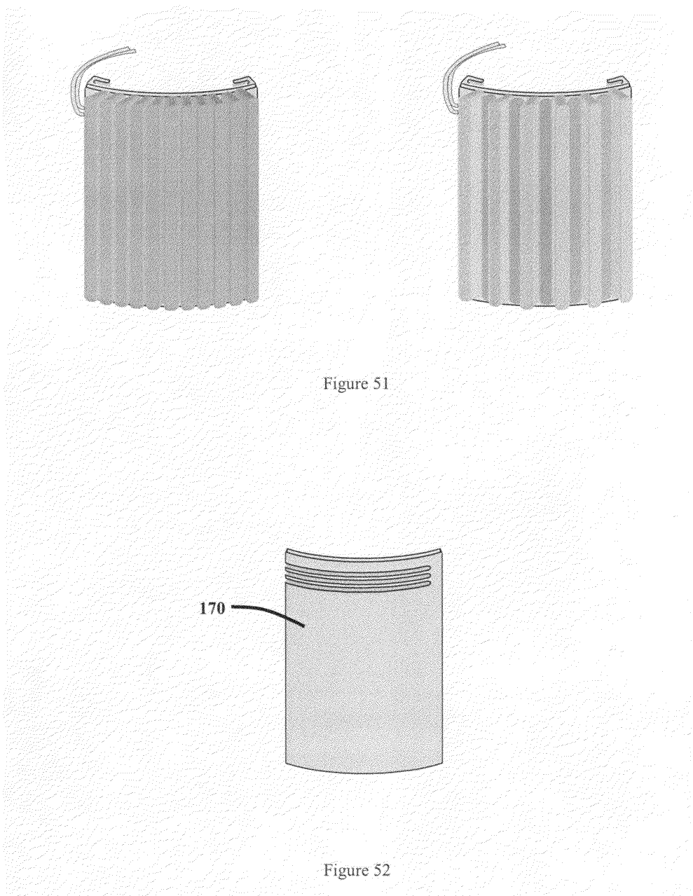

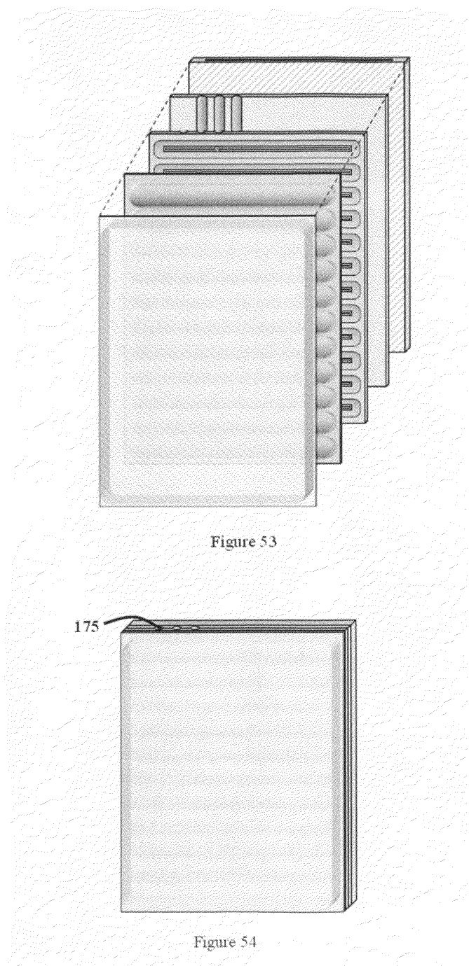

FIG. 49 illustrates a preferred embodiment of a two-state fluid-operated atraumatic retractor comprised of potentially moldable and/or vacuum-formed components that can be joined by any suitable bonding technique to form a complete assembly that may be directly attached to an existing appropriately sized surgical retractor. Three components in addition to two flexible umbilical tubes (or one two-section umbilical) 156 comprise this exemplary device; front-views of them are shown on the left-side of the figure and rear-views of them are shown on the right-side of the figure. The rearmost section which is shown at the tops of these columns of components has slots 157 within its front surface 155 that act as half of two fluid-conducting channels. When surface 155 is joined to the rear surface of the middle-positioned component, slots 160 become the second half of the two fluid-conducting channels, simultaneously forming a manifold having holes that conduct the fluid to the front surface of this middle-positioned component whereupon at the hole positions, slots 159 exists to channel the fluid throughout chambers that are formed when the front-most component, having flexible inelastic cavities 162 is attached and bonded to the front surface of the middle-positioned component at all regions bordering the cavities. Each of the slots 159 is scribed with narrow channels that intersect the holes to ensure free flow of the fluid throughout the chambers when they are collapsed to the extent that the inner surfaces of the cavities are pushed against the inner surfaces of the slots. When in a functional position, pressure from the retracted tissue collapses the cavities until they are internally pressurized to protrude and assume the appearance of the inflated cavity 161. Tabs 158 at the rear of the back surface, which may be full-length tabular constructions on each side or small tabs at various positions along each edge, acts as hooks that secure these flexible assemblies to existing retractor blades. FIG. 50 shows these components in a proper order of assembly. FIG. 51 shows fully assembled atraumatic retractors of this type, the drawing on the left depicting one with all chambers inflated and the drawing on the right depicting one with five of its eleven chambers deflated. FIG. 51 shows an example of the rearmost component of a three-state retractor that operates on the same principle.

Many of these fluid-operated embodiments may be produced to be consumable items, not meant for re-sterilization and reuse, although with the use of proper materials and assembly techniques, some of these models could be constructed to be reusable.

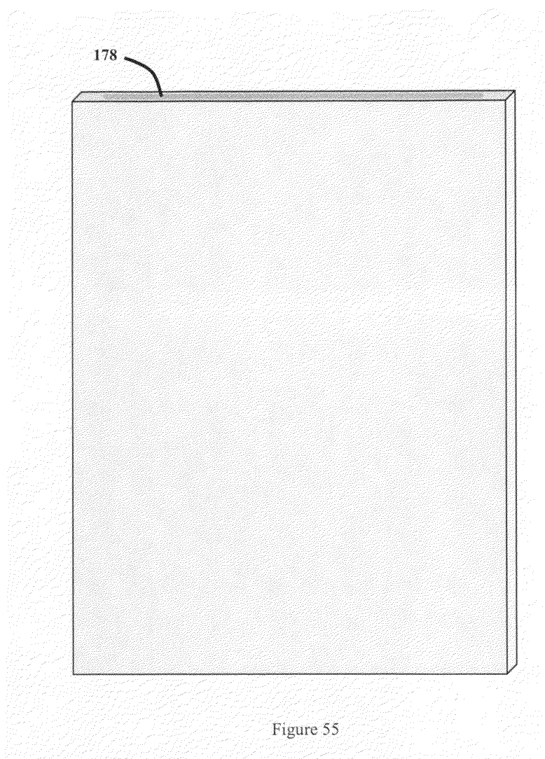

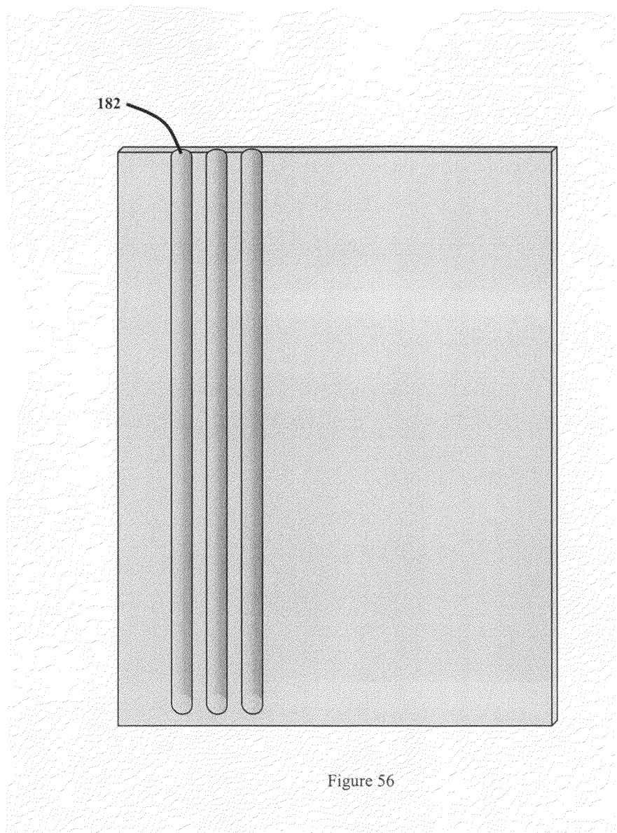

Already mentioned is the atraumatic surgical retraction and head-clamping device of FIG. 54 that employs fluid for its operation. FIG. 53 shows a similar assembly-guide of flexible materials to form a three-stage device which, in collaboration with two like-devices, is suitable for employment to stably position a patient's head during surgeries over many hours. In this embodiment, a rear back plate is included since it is not an item that is to be sandwiched between tissue and an existing retractor blade, and a front flexible and easily cleanable membrane is included to help prevent prepping solutions from settling in areas between the inflatable segments and drying. FIG. 54 shows these components assembled with the fluid channel openings 175 ready for attachment to an umbilical. FIG. 55 shows the rear component of a similar device modified for use as a three-state atraumatic retractor where, as in an earlier example, the device incorporates a pocket within a flexible layer that can accept a blade for easy attachment. FIG. 56 shows the layer incorporating slots 182 that, along with slots in the rear surface of the layer shown in FIG. 57 becomes the manifold that distributes fluid, this time to chambers oriented at an angle 90-degrees rotated with respect to the earlier example. The layer in FIG. 57 clearly shows its channels 186, its through-holes 187, and its grooves 185 that ensure distribution of the fluid throughout the chambers formed when this layer is bonded to the rear of its adjoining layer 190 shown in FIG. 58 to depict a similarly inelastic, flexible, cavity-containing layer with cavities 192 and sections 191 between cavities which, along with the remaining cavity-surrounding areas on the rear surface, this layer is bonded tightly to the previously shown layer. FIG. 59 shows an example of a flexible, cleanable cover-layer formed to have one large cavity that covers the full set of cavities of the previous layer. FIG. 60 shows this complete assembly.

FIG. 73 illustrates a proposed method of applying small amounts of retraction pressure to multiple parallel zones of delicate tissues, such as those within the brain, using acoustical power that can form standing waves within a flexible waveguide-confined liquid. With this means of generating peaks 222 and troughs 223 along an otherwise flat surface, the locations of the peaks can be made to continuously move along the retractor's length or switched to have changed positions as a function of the value of the driving frequency applied to one end of the waveguide by an ultrasonic transducer 221 powered through a small cable 220. Care must be taken to ensure that the temperature of the fluid in the resonantly driven waveguide is maintained within a safe range for the tissues that may be addressed.

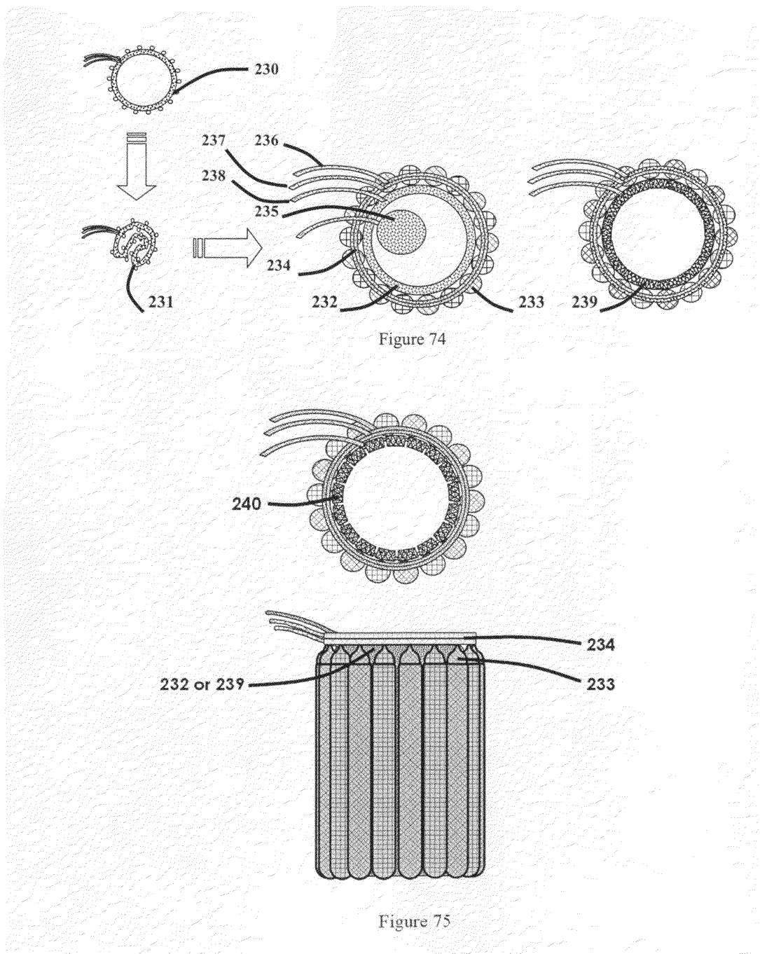

FIG. 74 and FIG. 75 illustrate a fluid-driven minimally invasive two-state retractor. It is designed to have a cylindrically shaped construction that is sufficiently thin and flexible to be folded into itself and inserted into an opening created by a small but appropriately deep incision. Two types of atraumatic minimally invasive retractors are displayed. One type, less robust than the other but less complicated to fabricate, employs a thin, flexible, inelastic, ring-shaped/long (i.e., thick-walled mailing-tube-shaped having length approximately equal to the depth of the retractor) inflatable chamber 232 that can inflate the atraumatic retractor from its center after being given “boost” assistance from a centrally inserted cylindrically shaped (almost pencil-thin) thick-walled balloon 235 (shown partially expanded) that can increase its diameter by a factor of ten without rupture. Chamber 232 provides moderate resultant forces directed radially outward from its outer surface to maintain wound expansion. A second type has a similar mailing-tube-shaped structure 239, of length equal to the inflatable chamber 232, which is comprised of multiple long, keystone-shaped inelastic inflatable segments 240 (17 in this example) having discontinuous star-shaped multiple-spoke truss-like full-length dividers that provide shape-forming tension between sections of their inner walls. The keystone-shaped segments apply lateral forces to adjacent keystone-shaped segments as they inflate to exert resultant forces, greater than those of the first type of minimally invasive retractor, directed radially outward from their widest sides to maintain wound expansion and to provide, or assist in providing wound expansion when such expansion again requires a “boost” from the thick-walled balloon 235. In all other ways, the two atraumatic minimally invasive retractors operate in the same fashion and are put-into-service in the following way. The retractor (preferably primed if liquid is the driving medium), after first being verified to be unpressurized, is opened to a circular form 230 before being collapsed into a narrow oval shape and folded into a form 231 where one of the long sides of the oval is tucked inward to meet the inside surface of the other long side of the oval. The retractor may be further folded in a similar way to additionally reduce the circumference of the form until it can be easily inserted into the incision which is to be held partly open with narrow hand-retractors, most conveniently with the aid of a surgical assistant. Once the retractor is inserted into the wound to a sufficient depth, fluid is pumped into umbilical 238 (to inflate ring-shaped chamber 232 or 239 as appropriate) and, if necessary, balloon 235 to inflate the retractor. Fluid is then pumped into umbilicals 236 and 237, which feed even-numbered and odd-numbered peripheral inflatable chambers, respectively, and is then carried by circular manifolds 234 to fully expand the peripheral inflatable chambers 233 (of which there are 18 in this example). Once the wound has been open for a short period of seconds to minutes, as required due to muscle relaxation and viscoelastic stabilization, balloon 235 (if used) may be removed and cycling may begin, preferably by automatic control, first with pressure released from umbilical 236 (as an arbitrary starting-point) for a desired dwell time (typically several minutes) and then reinstated to the previous inflation pressure after which, following a short dwell time (of preferably at least several seconds) pressure is released from umbilical 237 for a similar (typically several-minute) dwell time and then reinstated to its previous inflation pressure after which, following another short dwell time, this complete cycle is repeated, preferably by automatic control. When the procedure is finished, pressure is preferably released first from umbilicals 236 and 237 before it is released from umbilical 238 after which the retractor may be removed.

We claim:

1. An Atraumatic Surgical Retraction and Head-Clamping Device, for retracting or for clamping tissue, the device comprising:
A structure having at least one surface having at least two segments, each of which has a surface, the positions of which can be changed with respect to each other to change the physical pressure applied to at least a portion of retracted or clamped tissue;
Means for attaching said structure to either another structure that is substantially stable or an appendage that may be gripped; and,
Means for applying a force to at least a portion of at least one segment for changing the positions of the segments' surfaces relative to each other.

2. The Atraumatic Surgical Retraction and Head-Clamping Device of claim 1 wherein said force is controlled by and/or delivered through mechanical, pneumatic, and/or hydraulic devices.

3. The Atraumatic Surgical Retraction and Head-Clamping Device of claim 1 wherein said force is created by and/or controlled by human muscle action, and/or a change in fluid pressure, and/or a source of electrical energy.

4. The Atraumatic Surgical Retraction and Head-Clamping Device of claim 1 wherein said means for applying a force comprises at least one of one or more sheathed cables, one or more flexible tubes, power-conducting materials, and one or more transducers that convert one form of power to another.

5. The Atraumatic Surgical Retraction and Head-Clamping Device of claim 1 further comprising means for regulating the force.

6. The Atraumatic Surgical Retraction and Head-Clamping Device of claim 5 wherein said means for regulating the force is manual and/or automatic.

7. The Atraumatic Surgical Retraction and Head-Clamping Device of claim 5 wherein said means for regulating the force is through human interaction with the device and/or indirect human interaction with one or more sensing devices.

8. The Atraumatic Surgical Retraction and Head-Clamping Device of claim 5 wherein said means for regulating the force includes controlling devices.

9. The Atraumatic Surgical Retraction and Head-Clamping Device of claim 8 wherein said controlling devices are partly or wholly regulated by known or potentially relevant systemic parameters and/or parameters related to characteristics of the proximal tissue.

10. The Atraumatic Surgical Retraction and Head-Clamping Device of claim 1 wherein said segments are comprised of protrusions having substantially common-facing sides that controllably apply pressure to regions of supported or retracted tissues.

11. The Atraumatic Surgical Retraction and Head-Clamping Device of claim 10 wherein said pressures applied by said protrusions are substantially equalized.

12. The Atraumatic Surgical Retraction and Head-Clamping Device of claim 1 wherein the surfaces of said segments may exist as sections of at least one rotatable or transversally movable helically shaped component, or sections of an elastic material disposed between the supported or retracted tissue and said at least one rotatable or transversally movable helically shaped component.

13. The Atraumatic Surgical Retraction and Head-Clamping Device of claim 1 wherein the surfaces of said segments may exist as sections of a transversally movable flexible strip having at least one raised portion, or sections of an elastic material disposed between the supported or retracted tissue and said transversally movable flexible strip having at least one raised portion.

14. The Atraumatic Surgical Retraction and Head-Clamping Device of claim 2 wherein said segments exist as substantially groups of parallel fingers, at least two groups of which are attached to or integrated into separate arms on one side of a scissors-like device that are pivotally joined such that the fingers of one group are interleaved with the fingers of the other group to assume positions forward, behind, or in-line-with the second-group's fingers, and at least two other groups of which are similarly disposed to function similarly on at least one other to-some-degree opposing side of said scissors-like device.

15. The Atraumatic Surgical Retraction and Head-Clamping Device of claim 1 wherein said segments comprise sections of collapsible inelastic tubing and/or sections of expandable tubing that are at least partially covered by inflation-limiting fabric or other similarly inflation-limiting material.

16. The Atraumatic Surgical Retraction and Head-Clamping Device of claim 1 wherein said segments are comprised of sections of at least partially expandable and/or collapsible chambers comprised of or at least partly covered by inelastic material, inflation-limiting fabric, or other similarly inflation-limiting material.

17. The Atraumatic Surgical Retraction and Head-Clamping Device of claim 1 wherein an elastic material is disposed between the supported or retracted tissue and at least parts of sections of said segments.

18. The Atraumatic Surgical Retraction and Head-Clamping Device of claim 1 wherein the means of attachment to said another structure is an adjunctive or integrated hook or set of hooks that border at least one region of the tissue-positioning device.

19. The Atraumatic Surgical Retraction and Head-Clamping Device of claim 1 wherein at least one of said segments has perforations through which liquid and/or gas may flow to bathe at least one section of tissue.

20. The Atraumatic Surgical Retraction and Head-Clamping Device of claim 1 wherein said segments comprise the periphery of a substantially oval or cylindrical non-rigid apparatus which operates to hold-widened a minimally invasive surgical incision.

21. The Atraumatic Surgical Retraction and Head-Clamping Device of claim 1 wherein at least one of said segments presents at least one zone of reduced physical pressure and/or partial vacuum to stimulate bleeding and encourage blood perfusion continuance.

22. The Atraumatic Surgical Retraction and Head-Clamping Device of claim 1 wherein at least one of said segments presents at least one region at which thermal energy may be added or extracted.

23. The Atraumatic Surgical Retraction and Head-Clamping Device of claim 1 wherein at least one of said segments presents at least one region that can vibrate the tissue.
